SEQUENCE LISTING (14 sequences)

SEQ ID NO: 1 - DNA, 40 bp, synthetic construct
  source 1..40: mol_type = other DNA
agacgctctgggtcctcagcaatgatgtctgagcacacgt

SEQ ID NO: 2 - DNA, 40 bp, synthetic construct
  source 1..40: mol_type = other DNA
agccaaggcatcaggtgaagtactgactgcaggtcgtcta

SEQ ID NO: 3 - DNA, 20 bp, synthetic construct
  source 1..20: mol_type = other DNA
cattgggcacacacaatggt

SEQ ID NO: 4 - DNA, 25 bp, synthetic construct
  source 1..25: mol_type = other DNA
cgttgacagaaatattgcagcaaag

SEQ ID NO: 5 - DNA, 20 bp, synthetic construct
  source 1..20: mol_type = other DNA
actcatcctggatatcggca

SEQ ID NO: 6 - DNA, 18 bp, synthetic construct
  source 1..18: mol_type = other DNA
actcattgtccggggtcg

SEQ ID NO: 7 - DNA, 42 bp, synthetic construct
  source 1..42: mol_type = other DNA
tcggtgcgtgaactgcatagacatcagacatgcaggactcca

SEQ ID NO: 8 - DNA, 39 bp, synthetic construct
  source 1..39: mol_type = other DNA
ggagtacgcacagcgtactggccccggccattgtatcga

SEQ ID NO: 9 - DNA, 20 bp, synthetic construct
  source 1..20: mol_type = other DNA
tgatagactgccacctcagc

SEQ ID NO: 10 - DNA, 20 bp, synthetic construct
  source 1..20: mol_type = other DNA
attgggttcgacactacccc

SEQ ID NO: 11 - DNA, 20 bp, synthetic construct
  source 1..20: mol_type = other DNA
cggacattttgccttcacac

SEQ ID NO: 12 - DNA, 19 bp, synthetic construct
  source 1..19: mol_type = other DNA
ggcccagttggttgcatat

SEQ ID NO: 13 - DNA, 240 bp, synthetic construct
  source 1..240: mol_type = other DNA
cgaaaaagacacactcatcctggatatcggcagtgcgcctgccaggagaatgatgtctgagcacacgtaccattgtgtgtgcccaatgcgcagcgctgaggacccagagcgtctgctgtattatgccaggaagttagccaaggcatcaggtgaagtcgttgacagaaatattgcagcgaagatagacgacctgcagtcagtgatggcgaccccggacaatgagtcacggacattttgcct

SEQ ID NO: 14 - DNA, 300 bp, synthetic construct
  source 1..300: mol_type = other DNA
gatagacgacctgcagtcagtgatggcgaccccggacaatgagtcacggacattttgccttcacacagatcagacatgcaggactccagctgaggtggcagtctatcaggatgtctatgcagttcacgcaccgacttctttgtacttccaggcaatgaaaggagtacgcacagcgtactggattgggttcgacactaccccattcatgttcgatacaatggccggggcttatccaacatatgcaaccaactgggccgacgaacaggtgttgaaagccaggaacataggactgtgctccgc

(19) United States
(12) Patent Application Publication
(10) Pub. No.: US 2026/0049368 A1
(43) Pub. Date: Feb. 19, 2026

(54) ASSAYS FOR DETECTION OF MAYARO VIRUS AND METHODS OF DETECTION THEREOF

(71) Applicant: University of Florida Research Foundation, Inc., Gainesville, FL (US)

(72) Inventors: Zhonghui Hugh Fan, Gainesville, FL (US); Morteza Alipanahrostami, Gainesville, FL (US); John A. Lednicky, Gainesville, FL (US)

(21) Appl. No.: 19/104,480

(22) PCT Filed: Sep. 11, 2023
(86) PCT No.: PCT/US2023/073850

§ 371 Date: Feb. 18, 2025

Related U.S. Application Data
(60) Provisional application No. 63/406,997, filed on Sep. 15, 2022.

(51) Int. Cl. C12Q 1/70

(52) U.S. Cl. CPC C12Q 1/701

(57) ABSTRACT

The present disclosure provide for methods of detecting Mayaro virus in a sample, assays for the detection of the Mayaro virus, and Mayaro virus-specific primers. An assay of the present disclosure can include Mayaro virus (MAYV)-specific primers, such as a MAYV-specific forward inner primer, a MAYV-specific backward inner primer, a MAYV-specific forward primer, and a MAYV-specific backward primer. A method provided in the present disclosure of detecting MAYV in a sample can include adding an amount of the sample to a reaction mixture and amplifying the RNA in the sample using reverse transcription loop-mediated isothermal amplification.

### CLAIM OF PRIORITY TO RELATED APPLICATION

This application claims priority to co-pending U.S. Provisional application entitled “ASSAYS FOR DETECTION OF MAYARO VIRUS AND METHODS OF DETECTION THEREOF” having Ser. No. 63/406,997, filed on Sep. 15, 2022, which is entirely incorporated herein by reference.

### STATEMENT REGARDING FEDERALLY SPONSORED RESEARCH OR DEVELOPMENT

This invention was made with government support under Grant No. R01AI155735 and Grant No. R01AI158868, awarded by the National Institutes of Health. The government has certain rights in the invention.

### SEQUENCE LISTING

This application contains a sequence listing filed in ST.26 format entitled “222112_2020_Sequence_Listing” created on Aug. 25, 2023, having 13,787 bytes. The content of the sequence listing is incorporated herein in its entirety.

### BACKGROUND

Mayaro virus (MAYV) is a member of the genus Alphavirus that is transmitted by mosquitoes. Human infections with MAYV result in an acute febrile illness and other symptoms, similar to clinical presentation from other mosquito-borne viruses such as Chikungunya virus (CHKV), Dengue virus (DENV), and Zika virus (ZIKV), making it difficult to differentiate MAYV infection from those caused by other viruses. Epidemiological data shows that MAYV is an emerging virus worldwide. This challenge is particularly true in regions where all the four viruses are endemic, resulting in the need of a specific diagnosis. The overlap between MAYV infection and the current pandemic of coronavirus disease (COVID-19) further complicates the clinical diagnosis due to their similar clinical symptoms. Moreover, MAYV can mutate and has the potential to cause an epidemic. Therefore, there is a compelling need for the development of effective and specific detection methods that can provide accurate identification for MAYV, such as at the point-of-care (POC) for clinical management and transmission prevention.

### SUMMARY

In general, embodiments of the present disclosure provide for methods of detecting Mayaro virus in a sample, assays for the detection of the Mayaro virus, and Mayaro virus-specific primers.

An embodiment of the present disclosure provides for an assay used for the detection of the Mayaro virus (MAYV) comprising: MAYV-specific primers comprising a MAYV-specific forward inner primer, a MAYV-specific backward inner primer, a MAYV-specific forward primer, and a MAYV-specific backward primer. In an aspect, each of the MAYV-specific primers can be complementary to nucleic acids encoding the NS1 nonstructural protein of MAYV. The MAYV-specific forward inner primer can comprise a sequence having at least 90% sequence identity thereto the sequence AGACGCTCTGGGTCCTCAGCAATGATGTCTGAGCACACGT (SEQ ID NO:1). In another aspect, the MAYV-specific forward inner primer can comprise a sequence having at least 90% sequence identity thereto the sequence TCGGTGCGTGAACTGCATAGACATCAGACATGCAGGACTCCA (SEQ ID NO:7). The MAYV-specific backward inner primer can comprise a sequence having at least 90% sequence identity thereto the sequence AGCCAAGGCATCAGGTGAAGTACTGACTGCAGGTCGTCTA (SEQ ID NO: 2). In another aspect, the MAYV-specific backward inner primer can comprise a sequence having at least 90% sequence identity thereto the sequence GGAGTACGCACAGCGTACTGGCCCCGGCCATTGTATCGA (SEQ ID NO:8). The MAYV-specific forward primer can comprise a sequence having at least 85% sequence identity thereto the sequence ACTCATCCTGGATATCGGCA (SEQ ID NO:5). In another aspect, the MAYV-specific forward primer can comprise a sequence having at least 85% sequence identity thereto the sequence CGGACATTTTGCCTTCACAC (SEQ ID NO: 11). The MAYV-specific backward primer can comprise a sequence having at least 85% sequence identity thereto the sequence ACTCATTGTCCGGGGTCG (SEQ ID NO:6). In another aspect, the MAYV-specific backward primer comprises a sequence having at least 85% sequence identity thereto the sequence GGCCCAGTTGGTTGCATAT (SEQ ID NO: 12). In another aspect, at least one of the primers can comprise a modification, where the modification can be selected from a dye molecule, a conjugation molecule, a functional group, a molecular beacon, or a combination thereof.

In some embodiments, the assay can further comprise one or both of a MAYV-specific forward loop primer and a MAYV-specific backward loop primer. The MAYV-specific forward loop primer can comprise a sequence having at least 75% sequence identity thereto the sequence CATTGGGCACACACAATGGT (SEQ ID NO:3). In another aspect, the MAYV-specific forward loop primer comprises a sequence having at least 75% sequence identity thereto the sequence TGATAGACTGCCACCTCAGC (SEQ ID NO:9). The MAYV-specific backward loop primer can comprise a sequence having at least 75% sequence identity thereto the sequence CGTTGACAGAAATATTGCAGCAAAG (SEQ ID NO:4). In another aspect, the MAYV-specific backward loop primer can comprise a sequence having at least 75% sequence identity thereto the sequence ATTGGGTTCGACACTACCCC (SEQ ID NO: 10).

In some embodiments, the assay can further comprise a mixture that comprises one or more of an isothermal amplification buffer, a DNA polymerase, a reverse transcriptase, a concentrated primer mix, a deoxynucleotide triphosphate, MgSO₄, and nuclease-free water. The assay can further comprise Antarctic thermolabile uracil-DNA glycosylase and deoxyuridine triphosphate. In some aspects, the total volume of the mixture can range from about 1 μL to about 1000 μL. In another aspect, the total volume of the assay mixture can be about 25 μL. In an aspect, the assay can be a reverse transcription loop-mediated isothermal amplification (RT-LAMP) assay.

Another embodiment of the present disclosure provides for a method of detecting Mayaro virus in a sample, the method comprising: adding about 0.1 μL to about 10 ml of the sample to about 1 μL to about 1 mL of the RT-LAMP reaction mixture and amplifying RNA in the sample using RT-LAMP, where the sample contains from 1 to 1×10⁷ genomic copies of Mayaro virus RNA per reaction. The sample can be directly added to the RT-LAMP reaction mixture or be processed using various reagents or kits to obtain RNA first before being added to the RT-LAMP reaction mixture. The volume of the RT-LAMP reaction mixture can range from about 1 μL to about 1 mL. The RT-LAMP reaction mixture can be an RT-LAMP assay according to any of the embodiments of the assay discussed previously. Optionally, the RT-LAMP reaction mixture can be part of an assay performed either before or after performance of CRISPR. The method can further comprise adding about 0.1 μL to about 10 μL of 10×-10,000× concentrate SYBR green dye either before or after the amplification step and observing a color change indicating the presence of Mayaro virus in the sample. Additionally, the method can further comprise a color visualization indicator selected from SYTO 9, hydroxynaphthol blue, calcein, malachite green, leuco crystal violet, or phenol red. Additionally, the method can further comprise a turbidity indicator derived from magnesium pyrophosphate.

Other methods, system, devices, and features, and advantages will be or become apparent to one with skill in the art upon examination of the following drawings and detailed description. It is intended that all such additional compositions, apparatus, methods, features, and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 shows a picture of a real-time amplification device (RAD) including a flashlight-shaped microscope and 3D-printed chambers for a heater, battery, and integrated electrical circuit for temperature control. A scale bar of 2 cm is shown on the bottom left.

FIG. 2 shows the process flow of using SPD (sample preparation device) and RAD for virus detection at POC. In part a, a sample is loaded into the first reservoir of the buffer unit, followed by virus lysis, RNA binding and washing steps by sliding the mixing unit along the buffer unit. All buffers pass through the paper in the detection unit due to the vacuum created by pulling out the plunger of a syringe connected to the drain unit. In part b, the detection unit is separated from SPD, and the rRT-LAMP mix is added into the reaction well. In part c, the detection unit is placed in the seat of the heater chamber cap (FIG. 9) followed by rRT-LAMP.

FIG. 3A shows an rRT-LAMP assay of 5×10⁶ to 50 GEs MAYV RNA per reaction. All plots are an average of three replicates.

FIG. 3B shows a calibration curve between threshold time and MAYV concentration.

FIG. 3C shows RT-LAMP endpoint detection of 15, 10, and 5 GEs of MAYV RNA. The endpoint detection was carried out after 30 min RT-LAMP reactions. NTC stands for no-template control.

FIG. 3D shows that no cross-reactivity was observed for MAYV-targeted RT-LAMP assay when it was carried out against ZIKV, CHIKV, DENV-1, DENV-4, and a mixture containing these four viruses.

FIGS. 4A-4C show the endpoint detection of MAYV in whole blood samples using SPD. FIG. 4A shows pictures of the detection units under ambient light after RT-LAMP assay of whole blood samples spiked with cultured MAYV (viable virus) and blood samples without MAYV as negative controls (NB). FIG. 4B shows pictures of the same detection units in FIG. 4A, illuminated by a blue LED flashlight. FIG. 4C shows a gel electrophoresis image of the amplicons from each device in FIG. 4A. The first lane is a DNA ladder while other lanes are for samples in FIG. 4A.

FIGS. 5A-5C show the detection of MAYV in clinical samples using SPD. FIG. 5A shows pictures of the detection units under ambient light after RT-LAMP assay of three clinical samples containing MAYV, MAYV and DENV-1, and free of viruses, respectively, as indicated on the left. The NTC stands for no-template control. FIG. 5B shows pictures of the same detection units in FIG. 5A, illuminated by a blue LED flashlight. FIG. 5C shows gel electrophoresis image of the amplicons from each device. The first lane is a DNA ladder while other lanes are for clinical samples and NTC.

FIG. 6A shows real-time amplification curves obtained from the RAD shown in FIG. 1. MAYV RNA concentrations of 5×10⁶, 5×10⁴, 5×10², and 50 GEs were used in the rRT-LAMP assay. NTC stands for no-template control.

FIG. 6B shows a calibration curve of the rRT-LAMP assay between the threshold time and RNA amount of MAYV.

FIG. 7 shows amplification curves obtained from the RAD shown in FIG. 1. Two whole blood samples spiked with two different amounts of MAYV were tested. One replicate was carried out for each concentration. NTC stands for no-template control.

FIG. 8A-8E shows different views of a sample preparation device (SPD). FIG. 8A shows an exploded view of the SPD, which comprises a buffer unit, a mixing unit, a detection unit, and a drain unit. The buffer unit comprises 4 reservoirs for the storage of a lysis buffer (reservoir 1), a binding buffer (reservoir 2), and 2 washing buffers (reservoirs 3 and 4). In each funnel-shaped reservoir, one stainless-steel ball is placed at the bottom that functions as a valve, preventing buffers from flowing down until it is desired (the balls are above the reservoirs for visualization). In order to ensure that the ball remains secure to prevent any undesired movement or leakage during transportation, wax is employed to fix the ball in place. The process starts with melting a piece of wax (Akrowax™ 130, Akron, OH, USA) in a small beaker, immersing the ball into the melted wax, followed by placing the ball, coated with a thin layer of wax, in the reservoir where the wax solidifies, forming a bond that can be broken when needed. The mixing unit contains a well and a pin, and it slides along the buffer unit through the sliding tracks on both sides of the buffer unit. When the pin in the mixing unit is aligned with the ball, it breaks the wax bond and lifts the ball to open the valve, allowing the solution to flow down through the mixing unit and into the detection unit. The paper pad in the detection unit (2 cm×2 cm) is used to collect RNA for subsequent RT-LAMP. The drain unit is integrated to the detection unit and it is designed to speed up the reagent discharge by connecting a syringe to the exit tube. FIG. 8B shows valve actuation enabled by sliding the mixing unit to align the pin with the ball, causing the ball to be lifted up and reagents discharged into the mixing unit. FIG. 8C shows the cross-sectional view of the elements in FIG. 8B. FIG. 8D shows a photograph of all components in SPD. The buffer and mixing units were 3D-printed using polylactic acid (PLA). The detection unit was made from a paper pad attached to a polycarbonate well layer. The drain unit was made by casting PDMS (polydimethyl siloxane) into a 3D printed mold and curing at 100° C. for 1 hour. The mold included a reverse pattern of the drain unit. FIG. 8E shows a photograph of an assembled SPD, in which the mixing unit slides along with the buffer unit, lifts balls in the reservoirs, sequentially discharging the buffers stored in the reservoirs. The detection unit is attached to the bottom of the mixing unit and placed in the drain unit's chamber. The exit tube of the drain unit is connected to a syringe.

FIGS. 9A-9D show photographs of the RAD. FIG. 9A shows a 3D-printed stand with a base including three chambers for a heater, batteries, and an integrated electronic circuit as a temperature controller. Black polylactic acid (PLA) was used as print material. FIG. 9B shows 3D-printed caps for three chambers shown in FIG. 9A. A cavity is created in the cap of the heater chamber and the detection unit in FIG. 8 can fit there properly. FIG. 9C shows the same stand shown in FIG. 9A with all electronic components in the place, but without chamber caps. FIG. 9D shows the assembled RAD connected with a computer. On the computer screen was an image of two wells in the detection unit: one well for the negative control which is dark (indicated by dashed lines) while the other well for a sample that had a positive signal.

FIG. 10A shows a spectrum chart of dye SYTO 9, provided by the corresponding manufacturer.

FIG. 10B shows a spectrum chart of the flashlight-shaped microscope, provided by the corresponding manufacturer.

FIG. 11 shows the temperature profile of the RAD shown in FIG. 9A-9D. The temperature was measured using a T-type thermocouple that was embedded in the rRT-LAMP reaction mixture. The temperature reached 62.5° C. in 5-min., thus the heater was turned on to reach the temperature equilibrium at least 5 min. before the rRT-LAMP. The inset shows the temperature fluctuation in rRT-LAMP reaction mixture while assay was being performed; the temperature is within the range of 60-65° C. recommended for rRT-LAMP.

FIG. 12 shows photographs of a commercially available coffee mug used as a water bath for isothermal amplification in the format of endpoint detection. The mug is powered by a rechargeable battery and controlled using a smartphone App. The subsequent detection of the amplicons of RT-LAMP is achieved by adding 0.5 μL of 10,000× concentrate SYBR green dye at the end and the color change can be observed by the naked eye if MAYV is present. To enhance visualization, a blue LED flashlight is used to illuminate the reaction mixture, and fluorescence signal would be observed. A no-template control is included, as a negative control, in each test. The test results, i.e., the image of detection units, are captured using a smartphone camera and the presence/absence of the amplicons after RT-LAMP is confirmed by gel electrophoresis.

FIG. 13 shows sequences and location of two sets of rRT-LAMP primers designed for detecting Mayaro virus (MAYV). The genomic sequence of MAYV can be accessed from GenBank at the National Center for Biotechnology Information (NCBI) with an accession #DQ001069.1. The displayed sequences are SEQ ID NO: 13 and SEQ ID NO: 14.

FIGS. 14A-14B show rRT-LAMP of MAYV RNA using primer set #1 (FIG. 14A) or primer set #2 (FIG. 14B) using primer concentrations at the values recommended by NEB or at ½NEB. The amount of MAYV ranges from 5×10⁶ to 5×10³ genome equivalents (GEs). Each curve is an average of three replicates. The y-axis (ΔRₙ) is fluorescence signal obtained by the real-time PCR instrument. NTC stands for no-template control.

FIG. 15 shows the endpoint RT-LAMP assay for detection of 5×10⁶ to 5 GEs/μL of MAYV RNA. The endpoint detection was carried out after 30 min RT-LAMP reactions. NTC stands for no-template control.

FIG. 16 shows the detection of MAYV in whole blood using SPD. Pictures of the detection units under ambient light and blue LED after RT-LAMP assay of whole blood samples with 6 and 0.6 GEs/μL of MAYV and blood samples without MAYV as negative blood. At each test, 50 μL of the blood sample was used.

FIG. 17 shows the results of the RT-LAMP assay designed for detecting MAYV when a set of clinical samples were used. These samples include one from a Haitian patient infected with MAYV, one from a Venezuelan patient infected with both DENV-1 and MAYV, and one blood plasma from a Haitian free of viruses. A PCR machine was used in these experiments, and the incubation time was 30 min., followed by the endpoint detection.

FIG. 18A shows the real-time amplification curves for MAYV RNA at 5×10⁶ to 50 GEs using a real-time PCR machine. The NTC stands for no-template control.

FIG. 18B shows a calibration curve between the threshold time and the amount of MAYV. The threshold time was provided by the commercial PCR machine.

FIG. 19 shows amplification curves generated from the RAD shown in FIG. 1. Three different viruses of ZIKV [42], SARS-CoV-2 [30], and DENV-4 were used. one replicate was carried out for each concentration. The orange dots are raw data, and the lines are fitted curves. The fit curve was generated by averaging three data points. ½ NEB concentration of the primers were used to prepare the RT-LAMP reaction mixture for each virus.

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles. In the drawings, similar reference numerals between figures designate like or corresponding, but not necessarily the same, elements.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biomedical engineering, molecular biology, and the like, which are within the skill of the art.

The following examples are put forth so as to provide those of ordinary skill in the art with a complete disclosure and description of how to perform the methods and use the materials disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

As used herein, the following terms have the meanings ascribed to them unless specified otherwise. In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. patent law and the term is open-ended, allowing for the presence of more than that which is recited so long as basic or novel characteristics of that which is recited is not changed by the presence of more than that which is recited, but excludes prior art embodiments.

### General Discussion

In accordance with the purpose(s) of the present disclosure, as embodied and broadly described herein, embodiments of the present disclosure, in some aspects, relate to assays and primers for detecting Mayaro virus.

In general, embodiments of the present disclosure provide for methods of detecting Mayaro virus in a sample, assays for the detection of the Mayaro virus, and Mayaro virus-specific primers.

The present disclosure includes an assay for the detection of the Mayaro virus (MAYV). The assay can include MAYV-specific primers, such as a MAYV-specific forward inner primer, a MAYV-specific backward inner primer, a MAYV-specific forward loop primer, a MAYV-specific backward loop primer, a MAYV-specific forward primer, and a MAYV-specific backward primer. Advantageously, the primers have high specificity and do not demonstrate cross-reactivity with other tested mosquito-borne viruses (e.g., Zika, Dengue, and Chikungunya) and airborne viruses such as SARS-CoV-2. The assay can be used in RT-LAMP amplification or traditional RNA amplifications.

In some embodiments, the assay can include the following set of MAY-V specific primers: the MAYV-specific forward inner primer comprising the sequence AGACGCTCTGGGTCCTCAGCAATGATGTCTGAGCACACGT (SEQ ID NO:1) or a sequence at least 90% similar thereto; the MAYV-specific backward inner primer comprising the sequence AGCCAAGGCATCAGGTGAAGTACTGACTGCAGGTCGTCTA (SEQ ID NO:2) or a sequence at least 90% similar thereto; the MAYV-specific forward primer comprising the sequence ACTCATCCTGGATATCGGCA (SEQ ID NO:5) or a sequence at least 85% similar thereto; and the MAYV-specific backward primer comprising the sequence ACTCATTGTCCGGGGTCG (SEQ ID NO:6) or a sequence at least 85% similar thereto.

The assay provided above can further include one or both of: the MAYV-specific forward loop primer comprising the sequence CATTGGGCACACACAATGGT (SEQ ID NO:3) or a sequence at least 75% similar thereto; and the MAYV-specific backward loop primer comprising the sequence CGTTGACAGAAATATTGCAGCAAAG (SEQ ID NO:4) or a sequence at least 75% similar thereto.

In some embodiments, the primers share at least 75%, at least 85%, or at least 90% sequence identity with the above sequences.

In some embodiments, the assay can include the following set of MAYV specific primers: the MAYV-specific forward inner primer comprising the sequence TCGGTGCGTGAACTGCATAGACATCAGACATGCAGGACTCCA (SEQ ID NO:7) or a sequence at least 90% similar thereto; the MAYV-specific backward inner primer comprising the sequence GGAGTACGCACAGCGTACTGGCCCCGGCCATTGTATCGA (SEQ ID NO:8) or a sequence at least 90% similar thereto; the MAYV-specific forward primer comprising the sequence CGGACATTTTGCCTTCACAC (SEQ ID NO:11) or a sequence at least 85% similar thereto; and the MAYV-specific backward primer comprising the sequence GGCCCAGTTGGTTGCATAT (SEQ ID NO: 12) or a sequence at least 85% similar thereto.

The assay provided above can further include one or both of: the MAYV-specific forward loop primer comprising the sequence TGATAGACTGCCACCTCAGC (SEQ ID NO:9) or a sequence at least 75% similar thereto; and the MAYV-specific backward loop primer comprising the sequence ATTGGGTTCGACACTACCCC (SEQ ID NO:10) or a sequence at least 75% similar thereto;

In some embodiments, the primers share at least 75%, at least 85%, or at least 90% sequence identity with the above sequences.

In some embodiments, the primer set only contains a forward inner primer, a backward inner primer, a forward primer, and a backward primer, without either forward loop primers or backward loop primers, or without both forward loop primers and backward loop primers.

In some embodiments, two or more primers targeting different regions of the virus genome may be used in the same RT-LAMP reactions. In some embodiments, each of the MAYV-specific primers are complementary to nucleic acids encoding the NS1 nonstructural protein of MAYV.

In some embodiments, the assay can also include a mixture of one or more of an isothermal amplification buffer, a DNA polymerase, a reverse transcriptase, a concentrated primer mix, deoxynucleotide triphosphates, magnesium-ion-containing compounds such as MgSO₄, and nuclease-free water. Additionally, the assay may include a color visualization indicator such as SYTO 9, SYBR green, hydroxynaphthol blue, calcein, malachite green, leuco crystal violet, or phenol red, or a turbidity indicator derived from magnesium pyrophosphate. Additionally, the assay may include Antarctic thermolabile uracil-DNA glycosylase and deoxyuridine triphosphate for carryover prevention. Additionally, the assay may include betaine. In some embodiments the total volume of the mixture is about 1 μL to 1000 μL, including the primers.

In some embodiments, the assay is an RT-LAMP assay. In some embodiments, RT-LAMP is a part of the assay after other techniques such as Clustered Regularly Interspaced Short Palindromic Repeats (CRISPR). In other embodiments, RT-LAMP is a part of the assay before other techniques such as CRISPR. In other embodiments, the assay can be a LAMP assay or standard PCR. Advantageously, only about 0.2-5 μL or about 1 μL of sample per about 25 μL of the assay is sufficient for virus detection using RT-LAMP, where the sample contains from 1 to 1×10⁷ or about 5×10³ to 5×10⁶ genomic copies of Mayaro virus RNA per reaction.

Also provided are methods of detecting Mayaro virus in a sample, including: adding about 0.2 μL-5 μL of the sample to a 25-μL reaction mixture; or adding about 0.1 μL to about 10 mL of the sample to a larger volume (e.g., about 1 μL-1000 μL) of reaction mixture; and amplifying RNA in the sample using RT-LAMP. The sample can contain from about 5×10³ or about 1 to 5×10⁷ genomic copies of Mayaro virus RNA per reaction. In some embodiments, the sample can be directly added to the RT-LAMP assay. In other embodiments, the sample can be processed using various reagents or kits to obtain RNA before being added to the RT-LAMP assay. The method can be performed with or without sample preparation. In some embodiments the reaction mixture is part of an RT-LAMP assay as described above. The method can further include adding about 0.1 to 10-μL of 10×-10,000× concentrate SYBR green dye either before or after the amplification step to initiate a color change. Observing a color change can indicate the presence of Mayaro virus in the sample; the color change depends on the dye used and if an optical excitation source (e.g., LED light) is used. Other detection methods may be used, including lines similar to the pregnancy test strips, bands as in gel electrophoresis, or optical signals as in real time PCR.

In some embodiments, the primers as described herein can include one or more modifications. The modifications can include such as dye molecules (e.g., fluorescein), a conjugation molecule, functional groups (e.g., biotin), molecular beacons, and other modifications for a variety of other detection methods.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

### INTRODUCTION

Mayaro virus (MAYV), genus Alphavirus, is an RNA virus that is transmitted by mosquitoes [1-3]. Human infections with MAYV may result in Mayaro Fever, which is an acute febrile illness that is often accompanied by joint and muscle pain. The clinical presentation and course of MAYV is similar to that seen with another alphavirus, Chikungunya virus (CHKV); there are also similarities with the clinical presentation seen with arboviruses, Dengue viruses 1 to 4 (DENV1 to DENV4) and Zika virus (ZIKV), making it difficult to differentiate MAYV infection clinically from these other viruses [4-6]. MAYV has been identified as an emerging virus in the Americas, highlighting the need for development of effective detection methods for diagnostic evaluations [1, 7]. This challenge is particularly true in regions where the aforementioned viruses are endemic, resulting in the requirement of specific diagnosis at the point-of-care (POC) for clinical management and development of appropriate public health interventions.

The standard techniques for detection of MAYV infection include serological tests such as enzyme-linked immunosorbent assay (ELISA) [4, 6, 8-10] and molecular tests such as reverse transcription polymerase chain reactions (RT-PCR) [6, 8, 11-13]. Traditional serological tests are of limited value for detection of acute infections (due to time required to produce antibodies in the blood of patients), and there are potential issues with cross reactivity with other closely related viruses. Acute diagnosis of MAYV infections generally requires RT-PCR, but standard RT-PCR techniques can be time-consuming and require skilled personal and costly equipment. As a result, they are typically carried out in laboratories, making them impractical for diagnostics at the POC, and are not affordable in clinical setting with limited resources, such as those regions where MAYV infections are usually encountered. Antigen-test strips based on lateral flow assays, such as those commonly used for coronavirus disease 2019 (COVID-19), influenza, and other virus' diagnostic tests, are not yet available for MAYV. They also tend to be less sensitive than molecular tests.

Loop-mediated isothermal amplification (LAMP) has proven to be a rapid and sensitive diagnostic technique for the detection of various pathogens [14-21]. Compared with PCR, LAMP requires simpler thermal management due to the isothermal condition, is faster (˜30 min. versus 2 hours for PCR) and has similar sensitivity. LAMP requires 4-6 primers that target 6-8 distinct sequences, providing high specificity. For RNA viruses such as MAYV, reverse transcription LAMP (RT-LAMP) can be used. RT-LAMP has been demonstrated to detect a wide range of RNA viruses including CHIKV [22-24], DENV1 to DENV4 [22-26], Ebola virus [27], human immunodeficiency virus (HIV) [28, 29], influenza virus [30-32], severe acute respiratory syndrome coronavirus 2 (SARS-CoV-2) [30, 31, 33-40], and ZIKV [22-24, 41-43]. A few commercially available instruments based on LAMP or RT-LAMP also exist, including NINA from PATH, BioRanger from Diagenetix, and Lucira Check It from Lucira Health [44-46]

A few real-time LAMP-based POC platforms have been reported in the literature. Although they are well-designed, sample preparation was not included in their detection systems, either using RNA/DNA purified in laboratory or directly adding a sample to the reaction mixture. It has been shown that LAMP assay without sample preparation reduced the detection sensitivity [33] and increased the number of false negatives [47]. We have aimed to integrate sample preparation with real-time RT-LAMP (rRT-LAMP) for virus detection at POC.

In this work, we report the development of a highly sensitive, specific, and rapid rRT-LAMP assay for the detection of MAYV. Additionally, we have developed POC platforms for carrying out the rRT-LAMP assay.

To achieve this, we designed a sample preparation device that performs virus lysis and RNA enrichment/purification without requiring pipetting. This device follows similar concepts to our previously developed device and is capable of processing blood samples at the POC. Note that the previous device was done with colorimetric detection only, which was carried out at the end of 30 min amplification. The colorimetric detection gives only binary results: presence or absence of viruses of interest. The real-time POC detection platform, integrated with the sample preparation device, enables the quantitative detection of MAYV (i.e., virus burden) at the POC.

We have assessed the sensitivity and specificity of the rRT-LAMP assay and demonstrated the utility of our platforms using purified MAYV RNA, plasma containing virus, or blood samples containing virus. Our results indicate that the rRT-LAMP assay is specific and highly sensitive, and the POC platform has the potential to be used in the field, including in resource-limited regions.

### Materials and Methods

### Cell Culture for MAYV

MAYV strain Guyane was obtained from BEI Resources (Manassas, VA; Catalog. no. NR-49911) and propagated in Vero E6 cells, which were obtained from the American Type Culture Collection (ATCC, Manassas, VA). Cell culture took place at 37° C. and 5% CO₂ using advanced Dulbecco's essential medium (aDMEM) and 10% fetal bovine serum (FBS) as previously described [48].

After about 80% of the cells displayed virus-induced cytopathic effects, virus RNA was extracted from spent cell culture medium using a QIAamp Viral RNA Mini Kit (Qiagen, Valencia, CA, USA) following the manufacturer's instructions [49].

rRT-LAMP Mix

A solution of 25 μL of mixture was used for each RT-LAMP assay. The 25 μL of mixture contains 2.5 μL of 10× isothermal amplification buffer (ISO), 8 U Bst 2.0 WarmStart® DNA polymerase, 7.5 U WarmStart® RTx reverse transcriptase (RTx), 2.5 UL of 10× concentrated primer mix (PMX), 1.4 mM deoxynucleotide triphosphate (dNTPs), and 6 mM MgSO₄. To eliminate possible carryover contamination, 0.5 units of antarctic thermolabile uracil-DNA glycosylase (UDG) and 0.7 mM of deoxyuridine triphosphate (dUTP) were also added to the mixture [50, 51]. Nuclease-free water was then added to fill the 25 μL volume. The nuclease-free water and dNTPs were purchased from ThermoFisher (MA, USA). All other reagents were obtained from New England Biolabs (NEB, Ipswich, MA, USA). The final volume of each component for preparation of 25 μL RT-LAMP reaction mixture is tabulated Table 3. When real time detection was used, an appropriate dye (either SYBR green or SYTO 9) was added as detailed below.

TABLE 3
Components and their volumes used
in the 25-μL RT-LAMP mixture.

| | Components | | | | | | | | |
|---|---|---|---|---|---|---|---|---|---|
| | dNTP | ISO | PMX | MgSO4 | Bst | RTx | dUTP | UDG | Water |
| Vol- | 3.5 | 2.5 | 2.5 | 1.5 | 1 | 0.5 | 0.5 | 0.5 | 12.5 |
| ume | | | | | | | | | |
| (μL) | | | | | | | | | |

rRT-LAMP Using a Commercial Cycler

We used a QuantStudio-3 real-time PCR (qPCR) instrument to study the effects of the primer concentrations on the incubation time and to determine the detection limit of our rRT-LAMP assay. rRT-LAMP assays were carried out by adding 1 μL of purified RNA and 0.5 μL of 10× concentrate SYBR green I nucleic acid gel stain in dimethyl sulfoxide (ThermoFisher) to the 25 μL RT-LAMP reaction mixture. A no-template control (NTC) was included as a negative control in each test. The reactions were carried out at 62.5° C. for 60 min. The fluorescence signals obtained by the PCR machine were analyzed through the QuantStudio-3 (ThermoFisher). We carried out three replicates for each RNA concentration and the results presented in this work are the average of the three replicates.

### POC Sample Preparation Device

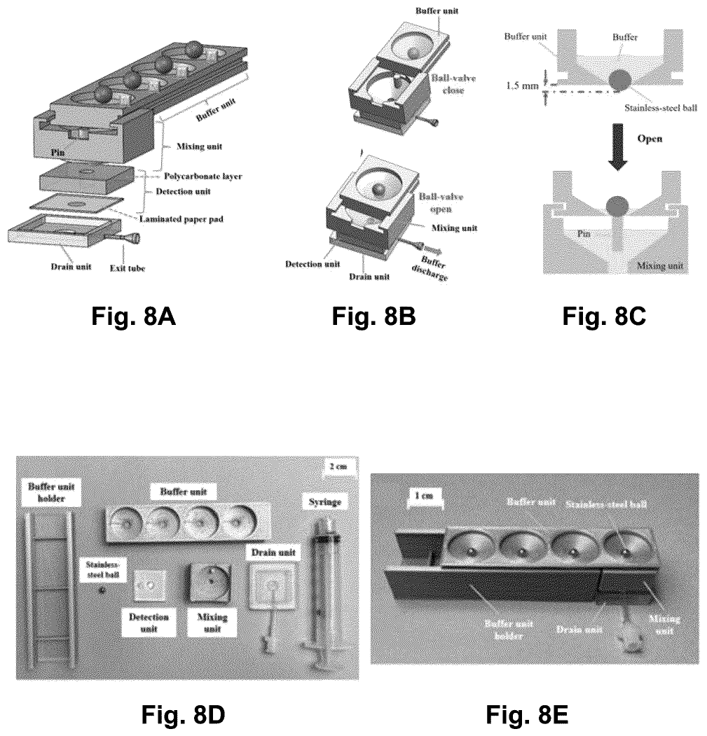

The sample preparation device (SPD) is an improved version of our previously developed device for the detection of ZIKV [42]. As shown in FIG. 8, SPD includes a buffer, mixing, detection, and drain units. The buffer and mixing units were 3D printed from polylactic acid (PLA). The detection unit is a paper-based amplification device including a polycarbonate well layer and a laminated paper pad. The laminated paper pad is a 4-mm Whatman chromatography paper (Fisher Scientific, Pittsburgh, PA, USA) that is sandwiched between two 75-μm-thick polyester lamination films. Chromatography paper was chosen in the device because it showed a lower limit of detection than FTA card and glass-fiber paper [52]. The SPD is developed to enable RNA purification and enrichment at the POC without the need for laboratory equipment. The RNA enrichment process in the previous device relied on the wicking effects of chromatography paper in the detection unit, which was adequate for processing low viscous samples such as saliva and urine. However, it took long time to process a viscous blood sample. To improve the device, we have developed a drain unit made of polydimethylsiloxane (PDMS), including a 1.9 cm×1.9 cm chamber for the detection unit and a 6 mm drains well in the center connected to a 1-mm drain channel (FIG. 8). This unit allows for the use of external forces such as a syringe to connect with the drain channel to discharge both the lysed sample and buffers quickly when needed. The size of the chamber was optimized to ensure PDMS's intrinsic sealing capability without the use of any sealant during the integration of the detection unit into the drain unit. The new design significantly reduced the sample preparation time to as low as 7 minutes (5-minute incubation for lysis and 2-minute flushing of all reagents in the buffer unit). A comparison between previously reported device (named VLEAD [42]) and SPD is presented in Table 2.

TABLE 2
Comparison between VLEAD and SPD.

| | VLEAD | SPD |
|---|---|---|
| Discharge mechanism | Wicking effects or | Syringe-induced vacuum |
| | capillary force | |
| Suitable samples | Low viscous samples | Both low and high |
| | such as saliva and | viscous samples such |
| | urine | as saliva, urine, and |
| | | blood |
| Discharge time using | ~3 hours | 2 minutes |
| blood samples | | |

### Real-Time Amplification Device

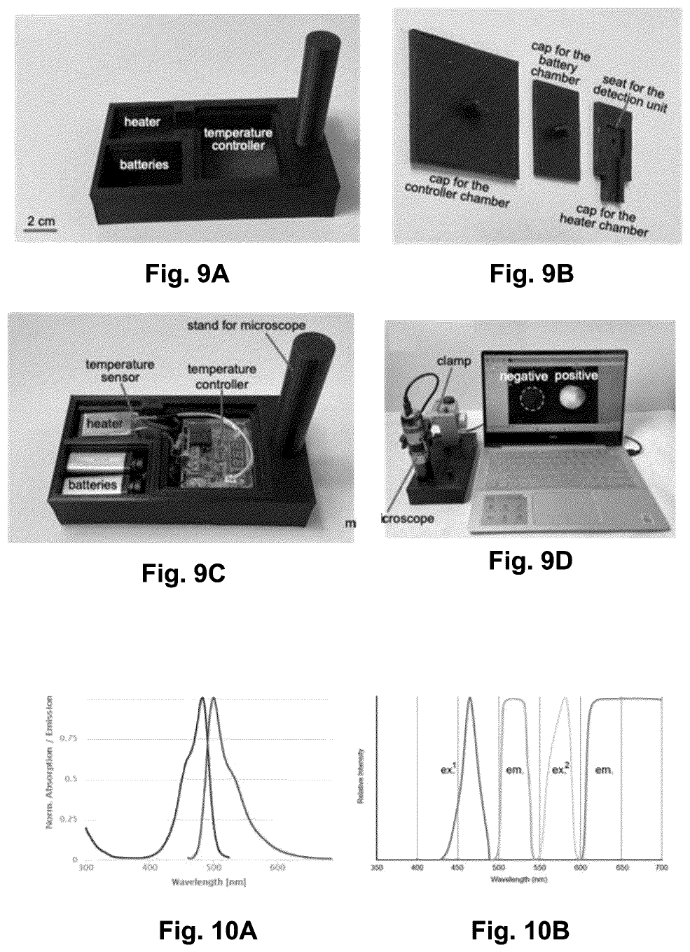

As shown in FIG. 1 and FIG. 9, our real-time amplification device (RAD) for rRT-LAMP at POC is composed of a portable stand, digital microscope (AM4117MT-G2FBW, Dino-Lite, USA), a temperature controller, a heater, and two 9-volt rechargeable batteries. The stand was 3D printed from PLA, with a base containing three chambers for the heater, batteries, and temperature controller. Three caps were also 3D-printed for covering the three chambers. The cap for the heater chamber was designed to contain a 2×2 cm² cavity, which properly fits the detection unit in FIG. 8 (which is also described later). In addition, two 5-mm holes were created in the cap, enabling real-time detection of the rRT-LAMP reactions (one hole for the sample and the other for the negative control).

The digital microscope possesses two bandpass filters which allow only desired excitation and emission wavelength to be transmitted through. The peak excitation wavelength is 465 nm while the emission band is 510-545 nm, as shown in FIG. 10B. These wavelengths are compatible with the excitation/emission wavelengths of SYTO 9 (FIG. 10A), which was used as fluorescent dye in our real-time detection platform. We replaced SYBR Green with SYTO 9 in the RAD since SYTO 9 has far less inhibitory effects on rRT-LAMP than SYBR Green [53]. We found that 4 UM of SYTO 9 is optimal for obtaining highest fluorescence intensity without inhibition on rRT-LAMP.

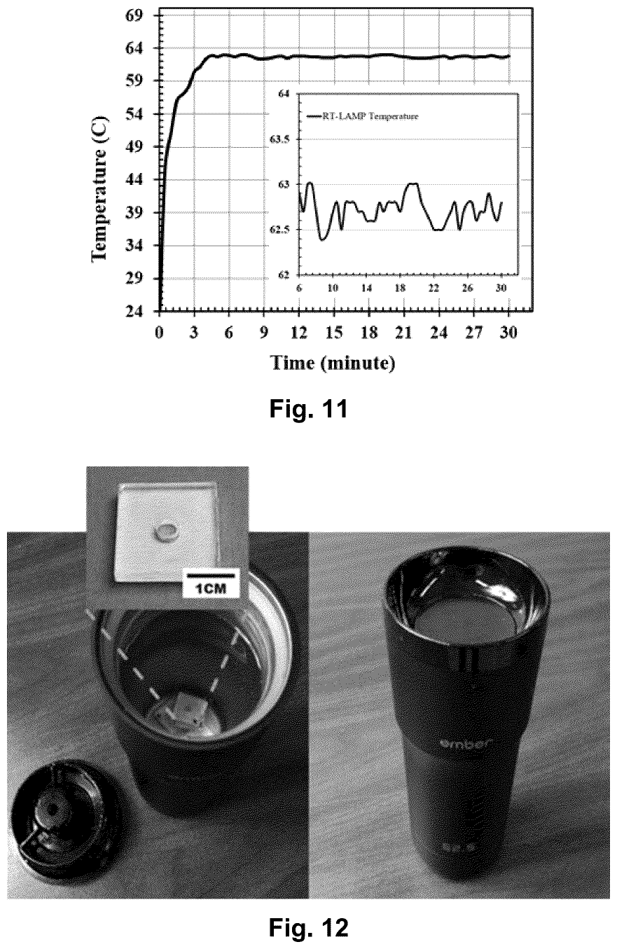

The heater used for rRT-LAMP is a positive temperature coefficient (PTC) heater (Bolsen Tech, USA), which can be powered by batteries, a laptop, or a smartphone. A commercially available, low-cost temperature controller was used to switch the PTC heater on or off. The controller's thermostat sensor was attached to the PTC heater and the sensor temperature was calibrated to set the rRT-LAMP reaction at 62.5° C. We characterized the heating system by placing a T-type thermocouple in the rRT-LAMP well and the resultant temperature profile is shown in FIG. 11. The results illustrate that the rRT-LAMP temperature was maintained between 62.5-63.1° C., which is within the standard temperature range (60-65° C.) recommended for LAMP. The batteries can last for about 3 hours, sufficient for at least 5 experiments. The total weight of RAD is 983 g, which is portable.

### Results and Discussion

### POC Testing

Detection can be performed at the end of the amplification reactions by direct visual observation of color change, which is known as endpoint detection. The endpoint detection is suitable for POC applications when the test can be performed for a specific, predetermined time, without the need for an expensive and complex detector. This method provides a binary result (“yes” or “no”), representing the presence or absence of a target virus, without any quantitative information. Alternatively, real-time detection can be employed to continuously monitor and analyze RT-LAMP, enabling dynamic observation of the RT-LAMP results. For positive samples, signal can be detected way before the predetermined time used for the endpoint detection, resulting in a much shorter analysis time. Additionally, this analysis time can be used to infer quantitative information about the amount of virus present in the sample tested because the less the amount of virus, the longer the amplification time required to have detectable signals.

We have employed the SPD and RAD for either endpoint detection or real-time detection of MAYV at the POC. To perform real-time detection of MAYV, we have integrated the SPD with RAD for sample preparation and real-time amplification. The SPD was designed for sample preparation and RNA enrichment at POC. After RNA purification on the paper pad of the detection unit, the detection unit of the SPD was separated from mixing unit and then placed in the cavity designed in the cap of the heater chamber in the RAD shown in FIG. 1 and FIG. 9. The digital microscope in FIG. 1 was programed to acquire fluorescence images every minute. The light-emitting diode (LED) in the microscope was programed to turn on for 5 s and off for 55 s to avoid possible photobleaching. Two Python-based App (one compatible with the computer operating system while the other with the smartphone operating system) were developed for measuring fluorescence intensity and displaying the data in real-time.

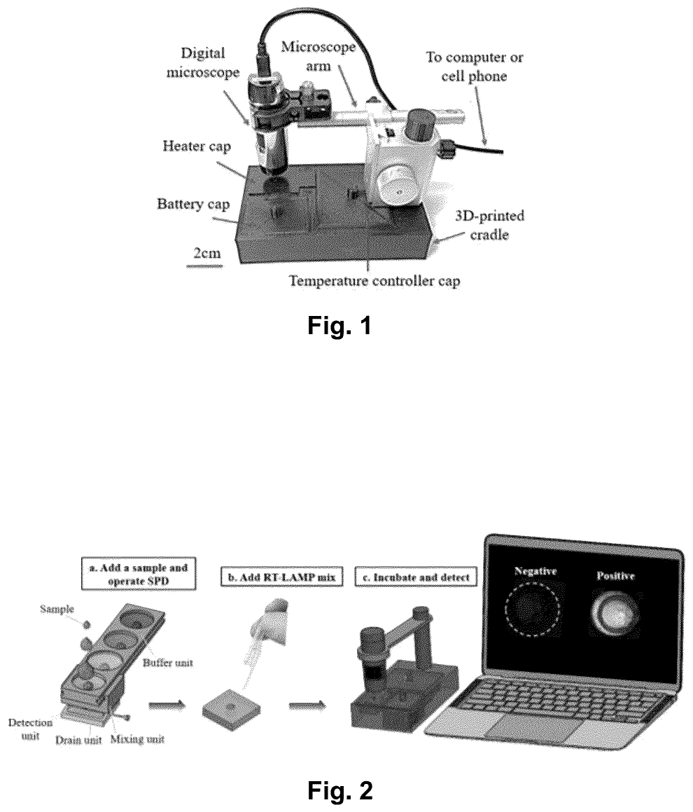

FIG. 2 shows the process flow of our portable pathogen detection platform. For the detection of MAYV, a sample (e.g., virus, plasma, or blood) is introduced to the lysis buffer in the first reservoir of the buffer unit (FIG. 2a). By sliding the mixing unit along the buffer unit (detailed in FIG. 8), the pin in the mixing unit pushes up the ball at the bottom of the first reservoir when they are aligned (FIG. 8B), discharging the solution in the reservoir from the buffer unit to the mixing unit and the detection unit. The discharge process is sped up by pulling the plunger of the syringe that is connected to the drain unit (FIG. 8). This ball-based valve design was inspired by a ballpoint pen, in which ink is dispensed onto paper when the metal ball at the tip is pressed while writing. Further sliding of the mixing unit along the buffer unit discharges the binding buffer in the second reservoir, resulting in retention of viral RNA on the paper pad when the solution in the mixing unit flows through the detection unit. The enriched RNA on the paper pad is then purified after two wash buffers in the third and fourth reservoirs are discharged in sequence by further sliding the mixing unit. After RNA purification, the detection unit is taken apart from SPD, followed by adding 25 μL rRT-LAMP mixture (FIG. 2b). Afterwards, the detection unit is placed in the cavity inside the cap of the heater chamber (while the temperature controller and the PTC heater have been switched on about 5 minutes earlier). The microscope is then positioned on top of the rRT-LAMP reaction well and takes pictures with 1 min intervals (FIG. 2c). The Python-coded software converts the images from the microscope into a graph.

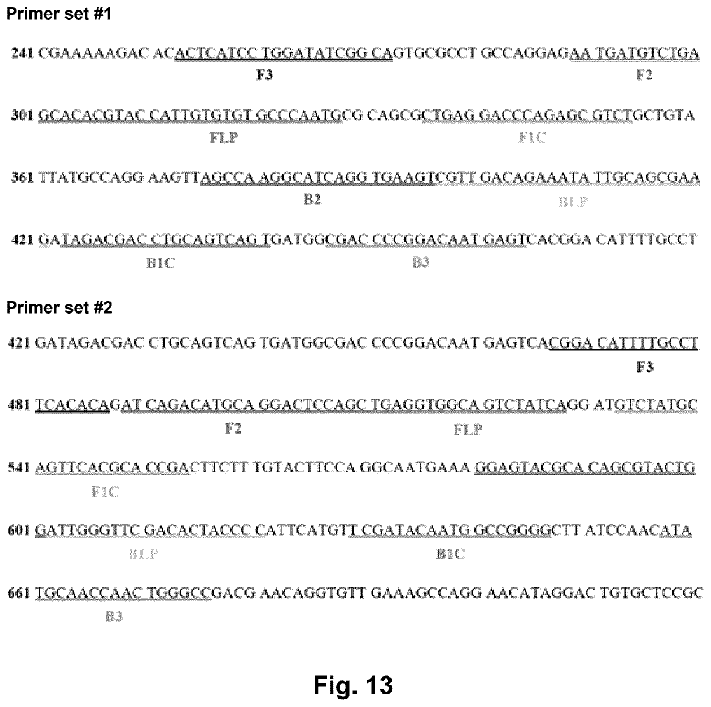

In some situations when binary detection is adequate, we performed isothermal amplification using endpoint detection. In this case, 25 μL RT-LAMP mixture is added into the detection unit, which is placed in a battery-powered coffee mug as illustrated in FIG. 12. The mug functions as an isothermal water bath incubator, enabling RT-LAMP at 62.5° C. for 30 min. The colorimetric detection is carried out at the end using SYBR Green dye; color change is observed by naked eye or recorded by a smartphone [42].

rRT-LAMP Assay

To design LAMP primers, we obtained the genome sequence of MAYV strain MAYLC from French Guiana from National Center for Biotechnology Information (NCBI, accession no. DQ001069). This virus strain was chosen as its genomic sequence has high identity with MAYV strains from Haiti and Venezuela that we have recently analyzed [48, 54], and thus serves as a contemporary reference strain for the type of MAYV we have tested in this work. Primer Explorer V5 program (https://primerexplorer.jp) was used to design the specific primers for MAYV under default control parameters. Two sets of rRT-LAMP primers were selected, targeting the conserved NS1 region of MAYV (FIG. 13). Each primer set can comprise forward inner primer (FIP), backward inner primer (BIP), forward loop primer (FLP), backward loop primer (BLP), forward external primer (F3), and backward external primer (B3), and their sequences are listed in Table 1 [14, 15].

TABLE 1
Sequences of the rRT-LAMP primers used for detection of MAYV RNA.

| | Primer set #1 sequences (5′→3′) | | Primer set #2 sequence (5′→3′) |
|---|---|---|---|
| F3 | ACTCATCCTGGATATCGGCA (SEQ | F3 | CGGACATTTTGCCTTCACAC (SEQ ID |
| | ID NO: 5) | | NO: 11) |
| B3 | ACTCATTGTCCGGGGTCG (SEQ ID | B3 | GGCCCAGTTGGTTGCATAT (SEQ ID |
| | NO: 6) | | NO: 12) |
| FIP | AGACGCTCTGGGTCCTCAGCAATGA | FIP | TCGGTGCGTGAACTGCATAGACATCA |
| | TGTCTGAGCACACGT (SEQ ID NO: 1) | | GACATGCAGGACTCCA (SEQ ID NO: 7) |
| BIP | AGCCAAGGCATCAGGTGAAGTACTG | BIP | GGAGTACGCACAGCGTACTGGCCCC |
| | ACTGCAGGTCGTCTA (SEQ ID NO: 2) | | GGCCATTGTATCGA (SEQ ID NO: 8) |
| FLP | CATTGGGCACACACAATGGT (SEQ | FLP | TGATAGACTGCCACCTCAGC (SEQ ID |
| | ID NO: 3) | | NO: 9) |
| BLP | CGTTGACAGAAATATTGCAGCAAAG | BLP | ATTGGGTTCGACACTACCCC (SEQ ID |
| | (SEQ ID NO: 4) | | NO: 10) |

Both primer set #1 and primer set #2 were checked for possible primer dimerization using Multiple Primer Analyzer from Thermo Fisher Scientific, and we found no problematic dimer formation among primers. Possible hairpin structure formation was analyzed using OligoAnalyzer from Integrated DNA Technologies (IDT); no hairpin structures were found to exist at 62.5° C., the reaction temperature that was used for rRT-LAMP in this work.

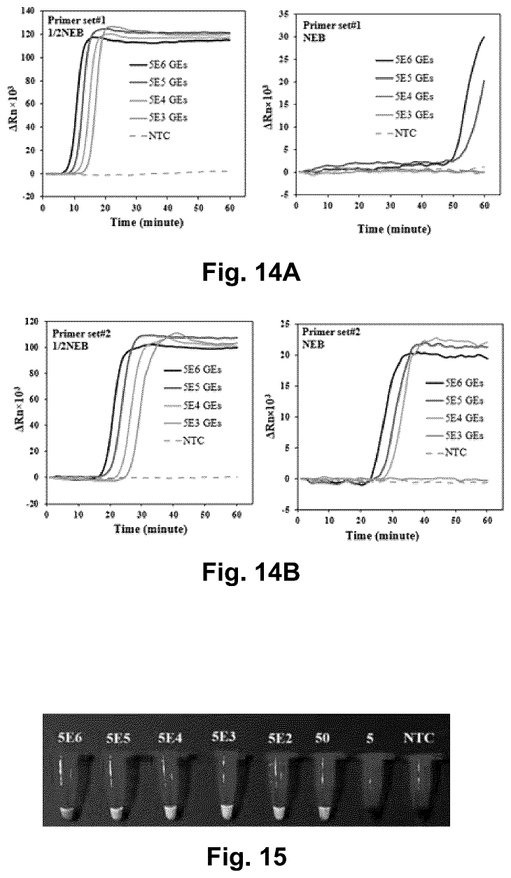

First, we employed the qPCR instrument to study the rRT-LAMP conditions such as assay time. We also used the qPCR instrument to compare primer set #1 with primer set #2 in Table 1 in terms of rRT-LAMP assay performance. In addition, we studied the effects of the primer concentrations on the assay performance since they are known to affect LAMP assays [55-57]. We compared the primer concentrations recommended by the LAMP kit manufacturer, New England Biolabs (NEB), with the primer concentrations at half the recommended concentrations, referred to as “½NEB”, based on our experience and the literature [30]. The concentrations of all primers used under these two conditions are detailed in Table 4. rRT-LAMP assays were carried out by using 1 μL RNA at concentrations from 5×10³ to 5×10⁶ GEs/μl. FIG. 14 shows rRT-LAMP curves using primer set #1 and #2. In either primer sets, inferior assay performance was obtained at NEB than at ½NEB. For example, for primer set #1, all four concentrations of viral RNA tested were detectable at ½NEB whereas only two highest concentrations (5×10⁶ GEs/μl and 5×105 GEs/μl) were detectable at NEB. In addition, although all four concentrations were detected using both primer sets under the ½NEB condition, the detection time with primer set #1 (threshold time of 8-14 min.) was shorter than primer set #2 (threshold time of 18-26 min.). Therefore, we chose primer set #1 and ½NEB primer concentrations for all subsequent experiments.

TABLE 4
Primer concentrations used in RT-LAMP

| Primer | NEB (μM) | ½NEB (μM) |
|---|---|---|
| F3 and B3 | 2 | 1 |
| FIP and BIP | 16 | 8 |
| FLP and BLP | 4 | 2 |

NEB: New England Biolabs, the kit manufacturer;
½NEB: at the half of the recommended values

### Assay Sensitivity and Specificity

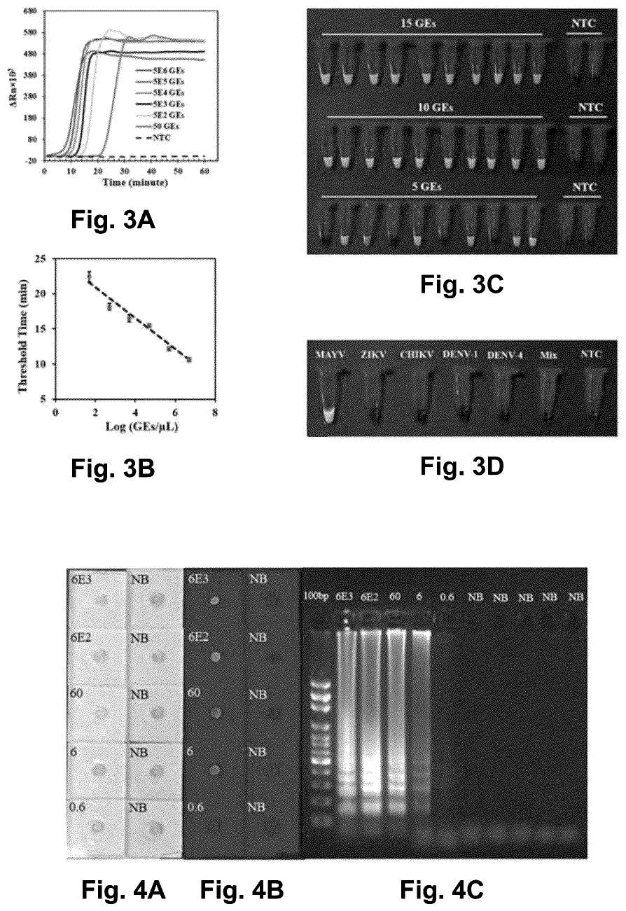

We investigated the limit of detection of the rRT-LAMP assay for MAYV detection. First, we used the qPCR machine to determine the required amplification time for low amount of viral RNA. FIG. 3a shows real time amplification curves of viral RNA ranging from 5×10⁶ GEs/μl to 50 GEs/μl. The results showed that all samples containing various concentrations of RNA reached a plateau within 30 min, indicating that 30 min RT-LAMP reaction is sufficient if we use the endpoint detection. The calibration curve between the threshold time (reported by the qPCR machine) and viral RNA concentration (FIG. 3b) indicates the feasibility of semiquantitative MAYV detection.

By fixing the amplification time at 30 min and not using the qPCR machine as an optical detector, we explored the colorimetric detection at the end. This method provides yes (presence of MAYV) or no (absence of MAYV) results observed with the naked eye. For this endpoint detection, 0.5 μL of 10000× concentrate SYBR green was added to the mixture after the reaction. We first performed the endpoint detection of serial dilutions of the MAYV RNA, ranging from 5 to 5×10⁶, to compare with the real-time detection results shown in FIG. 3a and determine the visual limit of detection of the assay. The results shown in FIG. 15 confirmed that the visual limit of detection of the 30 min RT-LAMP is between 50 and 5 GEs. To further narrow down the limit of detection of 30 min assay, we performed the endpoint detection in tube using 15, 10, and 5 GEs/μL of MAYV RNA. Ten experiments at each RNA concentration were conducted to ensure reproducibility of the assay and the results shown in FIG. 3c. The results in FIG. 3c show that all samples containing 10 GEs and 15 GEs of MAYV RNA were detected whereas only six out of 10 samples with 5 GEs were detected, indicating that visual limit of detection of our RT-LAMP assay is at least 10 GEs/reaction of MAYV RNA (i.e., 1 μL of 10 GEs/μL). This limit of detection is at least comparable to, if not better than, the limit of detection of 8.2 copies/μL with 5 μL of MAYV RNA (i.e., 41 copies total) using rRT-PCR [58].

To study the specificity of the RT-LAMP assay for MAYV detection, we tested it against four different mosquito-borne RNA viruses: CHIKV, two subtypes of DENV: DENV-1 and DENV-4, and ZIKV. Before experiments, we employed the basic local alignment search tool (BLAST) from NCBI to evaluate the similarities of the primer sequences designed for MAYV with the genomic sequences of the other four viruses. No similarities between the MAYV primers and sequences of other viruses were found. Isothermal amplifications were subsequently performed using the MAYV primers and each of the other viruses while the endpoint detection was used. For each test, 1 μL of 10⁶ GEs/μL virus RNA was tested per 25-μL RT-LAMP reaction. As shown in FIG. 3d, the RT-LAMP assay generated positive signals only for MAYV RNA, remaining negative for RNA of the other four viruses, their mixture, and the no-template control (NTC). The results indicate that there is no cross-reactivity between the MAYV RT-LAMP assay for the four mosquito-borne viruses tested.

### MAYV Endpoint Detection Using SPD

Our platform, which utilizes molecular amplification for the detection of MAYV, offers improved sensitivity and specificity compared to other LAMP-based POC platforms. This is due to the ability to process larger sample volumes (over 200 μL) than typical microfluidic platforms which can only handle a few microliters or less [59]. This leads to a lower detection limit, as more virus RNA can be enriched onto the detection unit's paper pad. Additionally, compared to those devices that benefit from the use of direct sample load, our detection system enables sample preparation and RNA purification at POC. It has been shown that LAMP assay without sample preparation reduced the detection sensitivity [33] and increased the number of false negatives [47].

A number of reports have documented erroneous outcomes in blood tests employing the LAMP method [60-64]. The LAMP technique has shown to generate false positive outcomes in turbidity assays due to the presence of inhibitors in the anticoagulant-containing blood samples [65] and protein precipitation [65, 66]. Unlike other POC LAMP-based devices developed for blood analysis in which the lysed blood is directly added to either RT-LAMP or LAMP reaction mixture [24, 67], our POC device eliminates all blood components by washing them out using washing buffers, as described above. This approach potentially eliminates false positive results stemming from blood components.

To carry out POC testing, we first employed SPD (FIG. 8) for sample preparation and used the MAYV RT-LAMP for the endpoint detection. Prior to the test, for 1 volume (e.g., 50 μL) of the sample, SPD was pre-loaded with 4 volume of lysis buffer (e.g., 200 μL), 4 volume of binding buffer (molecular biology grade ethanol), 4 volume of washing buffer 1 (AW1, QIAGEN) and 4 volume washing buffer 2 (AW2, QIAGEN), followed by sealing reservoirs using a thermoplastic film. The lysis buffer was made by mixing 1 volume of AVL (AVL, QIAGEN) and 1 volume of red blood cell lysis buffer (2.5 mM KHCO₃, 37.5 mM NH₄Cl, and 0.025 mM EDTA) (1×RBC lysis buffer, Thermo Fisher Scientific). Note that AVL was used for lysing viruses [30, 52] and 1×RBC was used for lysing red blood cells [68]. The operation of the SPD started with introducing 50 μL of MAYV sample. After a 5 min of incubation for sample lysis, the binding step was carried out by the sliding mechanism and ball-based valving incorporated in SPD as explained above. Washing steps were then performed in sequence by sliding between the buffer and mixing units. Enriched and purified RNA in the detection unit was subsequently subjected to 25 μL RT-LAMP and placed in a battery-powered coffee mug, for the endpoint detection. For the safety of the operator, virus samples were processed in a BSL-2+ (biosafety level) laboratory. The device, syringe, and others in contact with the virus sample were disposed of as biohazard waste.

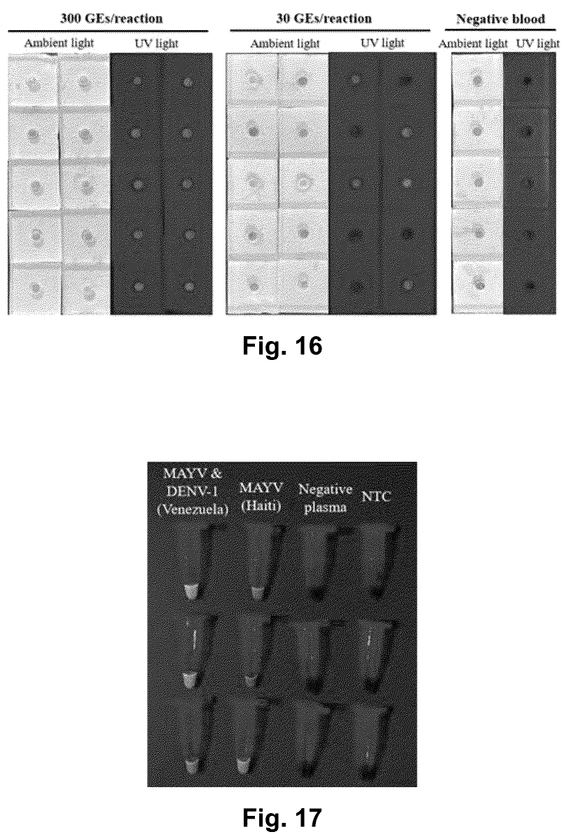

In order to assess the detection limit of our devices for sample-to-result testing at POC, we created whole blood samples by adding viable MAYV virus to healthy whole blood that were obtained from Innovative Research. The cultured virus used for spiking had a titer of 6×10⁴ GEs/μL. We then prepared 10-fold serial dilutions of this spiked sample using healthy blood to evaluate the device's performance. The SPD operation began by introducing 50 μL of the blood samples, followed by the SPD operation described above. As depicted in FIG. 4, we were able to detect MAYV from 6 GEs/μL (300 GEs/reaction), but no signals were observed for the 0.6 GEs/μL (30 GEs/reaction) and the negative blood control (NB, blood samples with no MAYV added). There were no false positives with the five negative blood samples tested. To ensure the robustness of our platform, ten replicates at two lowest MAYV amount were tested. As shown in FIG. 16, all samples containing 6 GEs/μL (300 GEs/reaction) were detected whereas only five samples with 0.6 GEs/μL (30 GEs/reaction) were detected, indicating that our POC system can detect at least 300 GEs/reaction in 37 min (7 min for sample preparation using SPD and 30 min for RT-LAMP reaction) when the endpoint detection is used. The detection limit of the device for blood samples spiked with cultured MAYV (viable viruses) is lower than the in-tube assay using purified MAYV RNA. The possible reasons include: (1) some RNA molecules might be lost during the processing, (2) inhibitory substances in blood samples might not be completely removed, thus having adverse effects on RT-LAMP assays, or (3) combination of both.

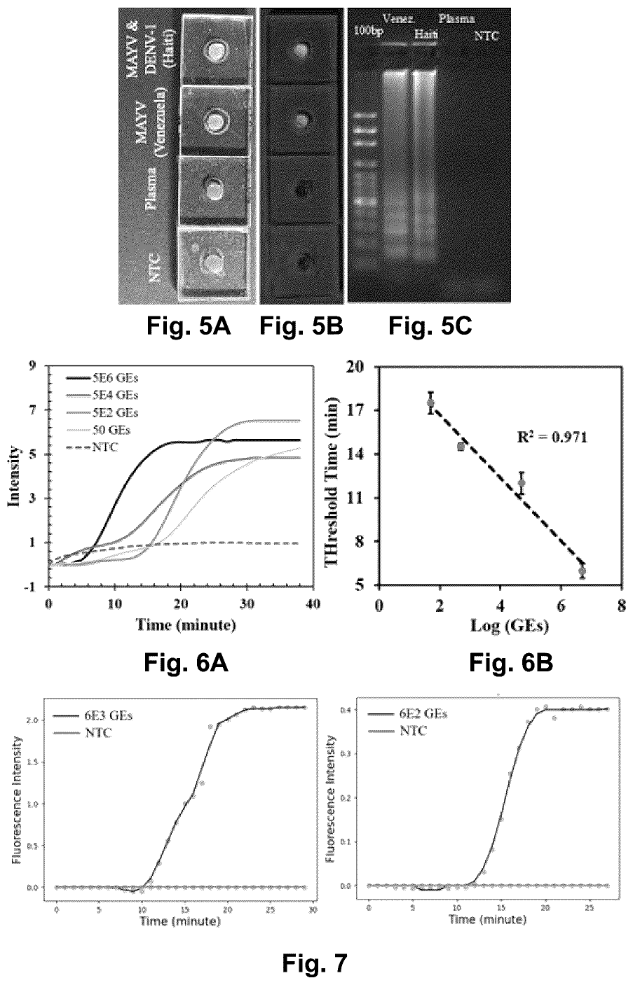

To evaluate our detection platform for detection of MAYV in clinical samples, we tested three patient plasma samples from either Haiti or Venezuela. These samples were collected according to protocols approved by US and country-specific Institutional Review Boards (IRB), as described previously [69]. One patient from Haiti was infected with MAYV and one from Venezuela had DENV-1 and MAYV infections [69]. The third plasma sample was from a Haitian patient that has tested free of mosquito-borne viruses and served as a negative control sample. The analyst performing the work was initially blinded as to the identity of these samples. RT-LAMP assays were first performed in tubes, with three replicate tests for each sample. Positive MAYV signals resulted for the Haiti and Venezuela samples, but no signals were generated for the negative-control plasma samples and the NTC samples (FIG. 17). We then performed the RT-LAMP assay for the detection of MAYV in these clinical samples using SPD by following the procedure explained above. Three replicates were carried out for each sample. Images of the detection unit for each sample are shown in FIGS. 5a and 5b. The RT-LAMP results were also confirmed by gel electrophoresis, shown in FIG. 5c. These results demonstrated that our RT-LAMP assay with SPD could detect MAYV at POC. The test results agree with those obtained by RT-LAMP assay in tubes (FIG. 17) and those obtained by standard RT-PCR [69].

### Real-Time Detection of MAYV

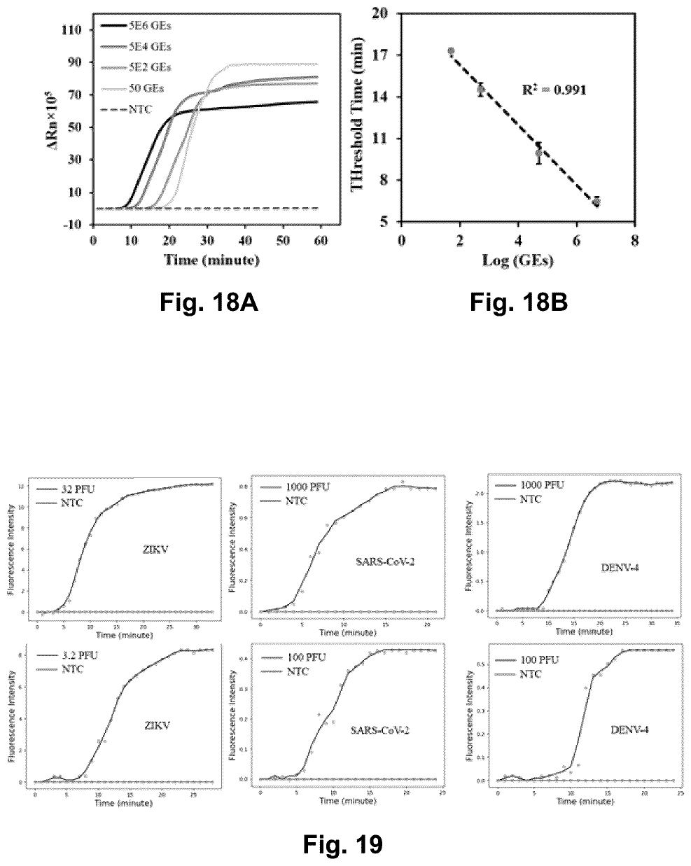

We developed a POC platform for rRT-LAMP, which was designed to assess the MAYV viral load at POC by combining the SPD (FIG. 8) with RAD (FIG. 1). We first used four MAYV RNA samples containing 5×10⁶, 5×10⁴, 5×10², and 50 GEs to evaluate the RAD. Each RNA sample was enriched using SPD, and the detection unit was then prepared for rRT-LAMP and placed in the heater chamber in FIG. 1. The amplification curves obtained from the flashlight-shaped digital microscope via the Python-based App are shown in FIG. 6a. The threshold time of each rRT-LAMP assay was calculated by the App, following the same definition as in real-time PCR (the time when the fluorescence signal reached 10 times the standard deviation of the baseline signals). We plotted the threshold time with the MAYV RNA amount, and a linear relationship between them was obtained as shown in FIG. 6b. This result indicates that our RAD system has the potential to provide quantitative information associated with MAYV virus load. We also performed rRT-LAMP assays of the same RNA samples using the commercial real-time PCR machine and the threshold times were given by the software associated with the PCR machine. The results of amplification curves and the calibration curves obtained from the PCR machine are shown in FIG. 18; they are comparable to that in FIG. 6, indicating that our POC platform is capable of providing with quantitative analysis of MAYV. The copy number of a gene of interest can be calculated from the threshold time using a standard curve [70]. This capability is crucial when/where the viral load of the virus in a patient needs to be monitored for clinical management and for taking appropriate measures to prevent virus transmission. FIG. 6 also depicts that the assay time was reduced to as low as 6 min if a positive signal was detected using the RAD. In other words, the time from the sample to result can be as short as 13 min. (7 min for sample preparation using SPD and 6 min for rRT-LAMP reaction using RAD).

To further evaluate our RAD for POC testing, we used whole blood samples spiked with cultured MAYV virus. Two virus loads of 6000 and 600 GEs/UL were tested. FIG. 7 shows the amplification curve generated by the app, showing the fluorescence intensity as a function of time for different concentrations of MAYV. These results indicate that our platform can be used to obtain the MAYV viral load in whole blood samples at POC.

To evaluate the feasibility of RAD for quantitative detection of other pathogens, we performed the real-time detection of ZIKV, DENV-4, and SARS-CoV-2 using RAD, and the amplification curves were shown in FIG. 19. The results demonstrated that our detection system can also detect other pathogens.

In our current real-time detection platform, the detection unit includes two reaction wells: one for processing a sample and the other for the negative control (NTC). However, it is possible to accommodate multiple samples by adjusting the microscope to have a larger field of view.

The cost of a POC test plays a crucial role in its potential commercialization. Each SPD costs $1.3 while the overall cost of reagents and the RT-LAMP mixture amounts to $4.45. Therefore, the total cost for a POC test using endpoint detection is $5.75, which is lower than other POC devices in the literature [44, 46, 71].

For real-time detection, however, there is one-time cost of RAD at $1003. Although the RAD cost is higher than other real-time platforms reported in the literature [38, 67], our system is reusable, capable of extending for other functions. Note that our real-time detection platform is still more than one order of magnitude cheaper than those commercially available real-time PCR machines.

### CONCLUSION

One of the main barriers for preventing and reducing virus-associated disease outbreaks, such as MAYV infection, is the lack of adequate tools for diagnosis, especially those providing fast and reliable detection at POC. Rapid testing is very crucial for clinical management and transmission reduction because the clinical symptoms of MAYV infection overlap with that of other mosquitoes-borne viruses such as CHIKV, DENV, and ZIKV.

LAMP is ideal for POC testing since it is an isothermal amplification technique, enabling it to be carried out using a constant heat source such as a heat block, water bath, and chemical reaction heat [73]. This advantage, as well as its faster reactions (30-min of endpoint detection versus 2 hours of PCR) and similar sensitivity, represents the key advantages of LAMP over the PCR-based methods.

Our team developed a rRT-LAMP assay for detecting MAYV and determined that its visual limit of detection was at least 10 GEs/reaction using MAYV RNA. The assay exhibited no cross-reactivity with CHIKV, DENV, or ZIKV. To enable detection of MAYV at POC, we incorporated the assay with SPD capable of detecting at least 300 GEs/reaction of MAYV in a blood sample. Additionally, we developed a portable RAD capable of quantitatively detecting MAYV and demonstrated its capacity for POC quantitative analysis of the virus. The results showed that the integrated devices have the potential for addressing challenges associated with infectious disease outbreaks caused by MAYV.

### REFERENCES

1. Pezzi L, Rodriguez-Morales A J, Reusken C B, Ribeiro G S, LaBeaud A D, Lourenco-de-Oliveira R, et al. GloPID-R report on chikungunya, o'nyong-nyong and Mayaro virus, part 3: Epidemiological distribution of Mayaro virus. Antiviral Res. 2019; 172:104610. Epub 2019 Sep. 24. doi: 10.1016/j.antiviral.2019.104610. PubMed PMID: 31545981.
2. Tesh R B, Watts D M, Russell K L, Damodaran C, Calampa C, Cabezas C, et al. Mayaro virus disease: an emerging mosquito-borne zoonosis in tropical South America. Clin Infect Dis. 1999; 28(1):67-73. doi: 10.1086/515070. PubMed PMID: 10028074.
3. de Thoisy B, Gardon J, Salas R A, Morvan J, Kazanji M. Mayaro virus in wild mammals, French Guiana. Emerg Infect Dis. 2003; 9(10):1326-9. doi: 10.3201/eid0910.030161. PubMed PMID: 14609474; PubMed Central PMCID: PMCPMC3033094.
4. Halsey E S, Siles C, Guevara C, Vilcarromero S, Jhonston E J, Ramal C, et al. Mayaro virus infection, Amazon Basin region, Peru, 2010-2013. Emerg Infect Dis. 2013; 19(11):1839-42. doi: 10.3201/eid1911.130777. PubMed PMID: 24210165; PubMed Central PMCID: PMCPMC3837653.
5. Friedrich-Janicke B, Emmerich P, Tappe D, Günther S, Cadar D, Schmidt-Chanasit J. Genome analysis of Mayaro virus imported to Germany from French Guiana. Emerg Infect Dis. 202014. p. 1255-7.
6. Pinheiro F P, Freitas R B, Travassos da Rosa J F, Gabbay Y B, Mello W A, LeDuc J W. An outbreak of Mayaro virus disease in Belterra, Brazil. I. Clinical and virological findings. Am J Trop Med Hyg. 1981; 30(3):674-81. doi: 10.4269/ajtmh. 1981.30.674. PubMed PMID: 6266263.
7. Acosta-Ampudia Y, Monsalve D M, Rodríguez Y, Pacheco Y, Anaya J M, Ramírez-Santana C. Mayaro: an emerging viral threat? Emerg Microbes Infect. 2018; 7(1):163. Epub 20180926. doi: 10.1038/s41426-018-0163-5. PubMed PMID: 30254258; PubMed Central PMCID: PMCPMC6156602.
8. Lima W G, Pereira R S, da Cruz Nizer W S, Brito J C M, Godói IP, Cardoso V N, et al. Rate of exposure to Mayaro virus (MAYV) in Brazil between 1955 and 2018: a systematic review and meta-analysis. Arch Virol. 2021; 166(2):347-61. Epub 20210107. doi: 10.1007/s00705-020-04889-9. PubMed PMID: 33410995.
9. Llagonne-Barets M, Icard V, Leparc-Goffart I, Prat C, Perpoint T, André P, et al. A case of Mayaro virus infection imported from French Guiana. J Clin Virol. 2016; 77:66-8. Epub 20160220. doi: 10.1016/j.jcv.2016.02.013. PubMed PMID: 26921736.
10. Cai Q, Mu J, Lei Y, Ge J, Aryee A A, Zhang X, et al. Simultaneous detection of the spike and nucleocapsid proteins from SARS-CoV-2 based on ultrasensitive single molecule assays. Anal Bioanal Chem. 2021; 413(18):4645-54. Epub 20210531. doi: 10.1007/s00216-021-03435-z. PubMed PMID: 34057558; PubMed Central PMCID: PMCPMC8166382.
11. Long K C, Ziegler S A, Thangamani S, Hausser N L, Kochel T J, Higgs S, et al. Experimental transmission of Mayaro virus by Aedes aegypti. Am J Trop Med Hyg. 2011; 85(4):750-7. doi: 10.4269/ajtmh.2011.11-0359. PubMed PMID: 21976583; PubMed Central PMCID: PMCPMC3183788.
12. Naveca F G, Nascimento V A D, Souza V C, Nunes B T D, Rodrigues D S G, Vasconcelos P. Multiplexed reverse transcription real-time polymerase chain reaction for simultaneous detection of Mayaro, Oropouche, and Oropouche-like viruses. Mem Inst Oswaldo Cruz. 2017; 112(7):510-3. doi: 10.1590/0074-02760160062. PubMed PMID: 28591313; PubMed Central PMCID: PMCPMC5452489.
13. Botes M, de Kwaadsteniet M, Cloete T E. Application of quantitative PCR for the detection of microorganisms in water. Anal Bioanal Chem. 2013; 405(1):91-108. Epub 20120925. doi: 10.1007/s00216-012-6399-3. PubMed PMID: 23001336; PubMed Central PMCID: PMCPMC7079929.
14. Notomi T, Okayama H, Masubuchi H, Yonekawa T, Watanabe K, Amino N, et al. Loop-mediated isothermal amplification of DNA. Nucleic acids research. 2000; 28(12):E63. PubMed PMID: 10871386; PubMed Central PMCID: PMCPMC102748.
15. Notomi T, Mori Y, Tomita N, Kanda H. Loop-mediated isothermal amplification (LAMP): principle, features, and future prospects. J Microbiol. 2015; 53(1):1-5. Epub 20150104. doi: 10.1007/s12275-015-4656-9. PubMed PMID: 25557475.
16. Nagamine K, Hase T, Notomi T. Accelerated reaction by loop-mediated isothermal amplification using loop primers. Mol Cell Probes. 2002; 16(3):223-9. doi: 10.1006/mcpr.2002.0415. PubMed PMID: 12144774.
17. Lin Q, Ye X, Yang B, Fang X, Chen H, Weng W, et al. Real-time fluorescence loop-mediated isothermal amplification assay for rapid and sensitive detection of Streptococcus gallolyticus subsp. gallolyticus associated with colorectal cancer. Anal Bioanal Chem. 2019; 411(26):6877-87. Epub 20190806. doi: 10.1007/s00216-019-02059-8. PubMed PMID: 31388715.
18. Ali H, Alkhaursi K, Holton T. Development of a colorimetric RT-LAMP assay for the detection of SARS-CoV-2 isolated from Oman. J Infect Dev Ctries. 2022; 16(6):952-8. Epub 20220630. doi: 10.3855/jidc. 15377. PubMed PMID: 35797288.
19. Shuryaeva A K, Malova T V, Tolokonceva A A, Karceka S A, Gordukova M A, Davydova E E, et al. Development and application of LAMP assays for the detection of enteric adenoviruses in feces. Microbiol Spectr. 2022; 10(4):e0051622. Epub 20220711. doi: 10.1128/spectrum.00516-22. PubMed PMID: 35862966; PubMed Central PMCID: PMCPMC9430467.
20. Kobayashi M, Mashiko T, Wilisiani F, Hartono S, Nishigawa H, Natsuaki T, et al. Development of a RT-LAMP assay for real-time detection of criniviruses infecting tomato. J Virol Methods. 2023; 312:114662. Epub 20221128. doi: 10.1016/j.jviromet.2022.114662. PubMed PMID: 36455691.
21. Jiang J, Feindel W, Swisher Grimm K, Harding M, Feindel D, Bajema S, et al. Development of a Loop-Mediated Isothermal Amplification (LAMP) Method to Detect the Potato Zebra Chip Pathogen ‘Candidatus Liberibacter solanacearum’ (Lso) and Differentiate Haplotypes A and B. Plant Dis. 2023; 107(6):1697-702. Epub 20230612. doi: 10.1094/pdis-09-22-2258-sr. PubMed PMID: 36480736.
22. Priye A, Bird S W, Light Y K, Ball C S, Negrete O A, Meagher R J. A smartphone-based diagnostic platform for rapid detection of Zika, chikungunya, and dengue viruses. Scientific reports. 2017; 7:44778. doi: 10.1038/srep44778. PubMed PMID: 28317856; PubMed Central PMCID: PMCPMC5357913.
23. Yaren O, Alto B W, Gangodkar P V, Ranade S R, Patil K N, Bradley K M, et al. Point of sampling detection of Zika virus within a multiplexed kit capable of detecting dengue and chikungunya. BMC Infect Dis. 2017; 17(1):293. doi: 10.1186/s12879-017-2382-0. PubMed PMID: 28427352; PubMed Central PMCID: PMCPMC5399334.
24. Ganguli A, Ornob A, Yu H, Damhorst G L, Chen W, Sun F, et al. Hands-free smartphone-based diagnostics for simultaneous detection of Zika, Chikungunya, and Dengue at point-of-care. Biomed Microdevices. 2017; 19(4):73. Epub 2017 Aug. 24. doi: 10.1007/s10544-017-0209-9. PubMed PMID: 28831630.
25. Lopez-Jimena B, Bekaert M, Bakheit M, Frischmann S, Patel P, Simon-Loriere E, et al. Development and validation of four one-step real-time RT-LAMP assays for specific detection of each dengue virus serotype. PLOS Negl Trop Dis. 2018; 12(5):e0006381. doi: 10.1371/journal.pntd.0006381. PubMed PMID: 29813062; PubMed Central PMCID: PMCPMC5973574 following competing interests: MBa and SF are employed by a commercial company, MAST Diagnostica GmbH, an industrial partner included in the EU-funded DiscoGnosis project.
26. Teoh B T, Sam S S, Tan K K, Johari J, Danlami M B, Hooi P S, et al. Detection of dengue viruses using reverse transcription-loop-mediated isothermal amplification. BMC Infect Dis. 2013; 13:387. Epub 20130821. doi: 10.1186/1471-2334-13-387. PubMed PMID: 23964963; PubMed Central PMCID: PMCPMC3846474.
27. Oloniniyi O K, Kurosaki Y, Miyamoto H, Takada A, Yasuda J. Rapid detection of all known ebolavirus species by reverse transcription-loop-mediated isothermal amplification (RT-LAMP). J Virol Methods. 2017; 246:8-14. Epub 20170327. doi: 10.1016/j.jviromet.2017.03.011. PubMed PMID: 28356221.
28. Curtis K A, Morrison D, Rudolph D L, Shankar A, Bloomfield L S P, Switzer W M, et al. A multiplexed RT-LAMP assay for detection of group M HIV-1 in plasma or whole blood. Journal of virological methods. 2018; 255:91-7. doi: 10.1016/j.jviromet.2018.02.012. PubMed PMID: 29474813.
29. Seok Y, Yin Q, Li R, Mauk M G, Bai H, Bau H H. Manually-operated, slider cassette for multiplexed molecular detection at the point of care. Sensors and actuators B, Chemical. 2022; 369:132353. PubMed PMID: 26900258.
30. Manzanas C, Alam M M, Loeb J C, Lednicky J A, Wu C Y, Fan Z H. A Valve-Enabled Sample Preparation Device with Isothermal Amplification for Multiplexed Virus Detection at the Point-of-Care. ACS Sens. 2021; 6(11):4176-84. Epub 2021 Nov. 13. doi: 10.1021/acssensors. 1c01718. PubMed PMID: 34767357; PubMed Central PMCID: PMCPMC8609915.
31. Rodriguez N M, Linnes J C, Fan A, Ellenson C K, Pollock N R, Klapperich C M. Paper-Based RNA Extraction, in Situ Isothermal Amplification, and Lateral Flow Detection for Low-Cost, Rapid Diagnosis of Influenza A (H1N1) from Clinical Specimens. Analytical chemistry. 2015; 87(15):7872-9. doi: 10.1021/acs.analchem.5b01594. PubMed PMID: 26125635; PubMed Central PMCID: PMCPMC4878390.
32. Jang M, Kim S, Song J. Rapid and simple detection of influenza virus via isothermal amplification lateral flow assay. Anal Bioanal Chem. 2022; 414(16):4685-96. Epub 20220502. doi: 10.1007/s00216-022-04090-8. PubMed PMID: 35501506; PubMed Central PMCID: PMCPMC9060413.
33. Dao Thi V L, Herbst K, Boerner K, Meurer M, Kremer L P, Kirrmaier D, et al. A colorimetric RT-LAMP assay and LAMP-sequencing for detecting SARS-CoV-2 RNA in clinical samples. Science translational medicine. 2020; 12(556). Epub 2020 Jul. 29. doi: 10.1126/scitranslmed.abc7075. PubMed PMID: 32719001; PubMed Central PMCID: PMCPMC7574920.
34. Garneret P, Coz E, Martin E, Manuguerra J C, Brient-Litzler E, Enouf V, et al. Performing point-of-care molecular testing for SARS-CoV-2 with RNA extraction and isothermal amplification. Plos One. 2021; 16(1):e0243712. Epub 2021 Jan. 12. doi: 10.1371/journal.pone.0243712. PubMed PMID: 33428641; PubMed Central PMCID: PMCPMC7799764.
35. Pang B, Xu J, Liu Y, Peng H, Feng W, Cao Y, et al. Isothermal Amplification and Ambient Visualization in a Single Tube for the Detection of SARS-CoV-2 Using Loop-Mediated Amplification and CRISPR Technology. Analytical chemistry. 2020; 92(24):16204-12. Epub 2020 Nov. 27. doi: 10.1021/acs.analchem.0c04047. PubMed PMID: 33238709; PubMed Central PMCID: PMCPMC7724759.
36. Rabe B A, Cepko C. SARS-CoV-2 detection using isothermal amplification and a rapid, inexpensive protocol for sample inactivation and purification. Proceedings of the National Academy of Sciences of the United States of America. 2020; 117(39):24450-8. Epub 2020 Sep. 10. doi: 10.1073/pnas.2011221117. PubMed PMID: 32900935; PubMed Central PMCID: PMCPMC7533677.
37. Zhang C, Zheng T, Wang H, Chen W, Huang X, Liang J, et al. Rapid One-Pot Detection of SARS-CoV-2 Based on a Lateral Flow Assay in Clinical Samples. Analytical chemistry. 2021; 93(7):3325-30. Epub 2021 Feb. 12. doi: 10.1021/acs.analchem.0c05059. PubMed PMID: 33570399; PubMed Central PMCID: PMCPMC7885334.
38. Panpradist N, Kline E C, Atkinson R G, Roller M, Wang Q, Hull I T, et al. Harmony COVID-19: A ready-to-use kit, low-cost detector, and smartphone app for point-of-care SARS-CoV-2 RNA detection. Sci Adv. 2021; 7(51):eabj1281. Epub 20211215. doi: 10.1126/sciadv.abj1281. PubMed PMID: 34910507; PubMed Central PMCID: PMCPMC8673764.
39. Ganguli A, Mostafa A, Berger J, Aydin M Y, Sun F, Ramirez S A S, et al. Rapid isothermal amplification and portable detection system for SARS-CoV-2. Proceedings of the National Academy of Sciences of the United States of America. 2020; 117(37):22727-35. Epub 2020 Sep. 2. doi: 10.1073/pnas.2014739117. PubMed PMID: 32868442; PubMed Central PMCID: PMCPMC7502724.
40. Jhou Y R, Wang C H, Tsai H P, Shan Y S, Lee G B. An integrated microfluidic platform featuring real-time reverse transcription loop-mediated isothermal amplification for detection of COVID-19. Sensors and actuators B, Chemical. 2022; 358:131447. Epub 2022 Feb. 1. doi: 10.1016/j.snb.2022.131447. PubMed PMID: 35095200; PubMed Central PMCID: PMCPMC8789398.
41. Song J, Mauk M G, Hackett B A, Cherry S, Bau H H, Liu C. Instrument-Free Point-of-Care Molecular Detection of Zika Virus. Analytical chemistry. 2016; 88(14):7289-94. doi: 10.1021/acs.analchem.6b01632. PubMed PMID: 27306491; PubMed Central PMCID: PMCPMC4955015.
42. Jiang X, Loeb J C, Manzanas C, Lednicky J A, Fan Z H. Valve-Enabled Sample Preparation and RNA Amplification in a Coffee Mug for Zika Virus Detection. Angew Chem Int Ed Engl. 2018; 57(52):17211-4. doi: 10.1002/anie.201809993. PubMed PMID: 30358036.
43. Calvert A E, Biggerstaff B J, Tanner N A, Lauterbach M, Lanciotti R S. Rapid colorimetric detection of Zika virus from serum and urine specimens by reverse transcription loop-mediated isothermal amplification (RT-LAMP). PLOS One. 2017; 12(9):e0185340. Epub 20170925. doi: 10.1371/journal.pone.0185340. PubMed PMID: 28945787; PubMed Central PMCID: PMCPMC5612724.
44. Mohon A N, Lee L D, Bayih A G, Folefoc A, Guelig D, Burton R A, et al. NINA-LAMP compared to microscopy, RDT, and nested PCR for the detection of imported malaria. Diagn Microbiol Infect Dis. 2016; 85(2):149-53. Epub 2016 Mar. 28. doi: 10.1016/j.diagmicrobio.2015.11.009. PubMed PMID: 27017271; PubMed Central PMCID: PMCPMC4862928.
45. Diaz L M, Johnson B E, Jenkins D M. Real-time optical analysis of a colorimetric LAMP assay for SARS-CoV-2 in saliva with a handheld instrument improves accuracy compared with endpoint assessment. J Biomol Tech. 2021; 32(3):158-71. Epub 2022 Jan. 15. doi: 10.7171/jbt.21-3203-011. PubMed PMID: 35027873; PubMed Central PMCID: PMCPMC8730521.
46. Zahavi M, Rohana H, Azrad M, Shinberg B, Peretz A. Rapid SARS-CoV-2 Detection Using the Lucira Check It COVID-19 Test Kit. Diagnostics (Basel). 2022; 12(8). Epub 2022 Aug. 27. doi: 10.3390/diagnostics 12081877. PubMed PMID: 36010227; PubMed Central PMCID: PMCPMC9406928.
47. Moon Y J, Lee S Y, Oh S W. A Review of Isothermal Amplification Methods and Food-Origin Inhibitors against Detecting Food-Borne Pathogens. Foods. 2022; 11(3). Epub 20220124. doi: 10.3390/foods11030322. PubMed PMID: 35159473; PubMed Central PMCID: PMCPMC8833899.
48. Blohm G M, Marquez-Colmenarez M C, Lednicky J A, Bonny T S, Mavian C, Salemi M, et al. Isolation of Mayaro Virus from a Venezuelan Patient with Febrile Illness, Arthralgias, and Rash: Further Evidence of Regional Strain Circulation and Possible Long-Term Endemicity. Am J Trop Med Hyg. 2019; 101(6):1219-25. Epub 2019 Oct. 10. doi: 10.4269/ajtmh. 19-0357. PubMed PMID: 31595869; PubMed Central PMCID: PMCPMC6896866.
49. Lednicky J, De Rochars V M, Elbadry M, Loeb J, Telisma T, Chavannes S, et al. Mayaro Virus in Child with Acute Febrile Illness, Haiti, 2015. Emerg Infect Dis. 2016; 22(11):2000-2. doi: 10.3201/eid2211.161015. PubMed PMID: 27767924; PubMed Central PMCID: PMCPMC5088037.
50. Longo M C, Berninger M S, Hartley J L. Use of uracil DNA glycosylase to control carryover contamination in polymerase chain reactions. Gene. 1990; 93(1):125-8. doi: 10.1016/0378-1119(90)90145-h. PubMed PMID: 2227421.
51. Hsieh K, Mage P L, Csordas A T, Eisenstein M, Soh H T. Simultaneous elimination of carryover contamination and detection of DNA with uracil-DNA-glycosylase-supplemented loop-mediated isothermal amplification (UDG-LAMP). Chem Commun (Camb). 2014; 50(28):3747-9. Epub 20140228. doi: 10.1039/c4cc00540f. PubMed PMID: 24577617.
52. Jiang X, Loeb J C, Pan M, Tilly T B, Eiguren-Fernandez A, Lednicky J A, et al. Integration of sample preparation with RNA-Amplification in a hand-held device for airborne virus detection. Anal Chim Acta. 2021; 1165:338542. Epub 20210423. doi: 10.1016/j.aca.2021.338542. PubMed PMID: 33975694; PubMed Central PMCID: PMCPMC8325109.
53. Monis P T, Giglio S, Saint C P. Comparison of SYTO9 and SYBR Green I for real-time polymerase chain reaction and investigation of the effect of dye concentration on amplification and DNA melting curve analysis. Anal Biochem. 2005; 340(1):24-34. doi: 10.1016/j.ab.2005.01.046. PubMed PMID: 15802126.
54. White S K, Mavian C, Elbadry M A, Beau De Rochars V M, Paisie T, Telisma T, et al. Detection and phylogenetic characterization of arbovirus dual-infections among persons during a chikungunya fever outbreak, Haiti 2014. PLOS Negl Trop Dis. 2018; 12(5):e0006505. Epub 2018 Jun. 1. doi: 10.1371/journal.pntd.0006505. PubMed PMID: 29851952; PubMed Central PMCID: PMCPMC5997359.
55. Liu J, Xu L, Guo J, Chen R, Grisham M P, Que Y. Development of loop-mediated isothermal amplification for detection of Leifsonia xyli subsp. xyli in sugarcane. Biomed Res Int. 2013; 2013:357692. Epub 20130423. doi: 10.1155/2013/357692. PubMed PMID: 23710444; PubMed Central PMCID: PMCPMC3655484.
56. Yeh H Y, Shoemaker C A, Klesius P H. Evaluation of a loop-mediated isothermal amplification method for rapid detection of channel catfish Ictalurus punctatus important bacterial pathogen Edwardsiella ictaluri. J Microbiol Methods. 2005; 63(1):36-44. Epub 20050331. doi: 10.1016/j.mimet.2005.02.015. PubMed PMID: 16157211.
57. Foo P C, Nurul Najian A B, Muhamad N A, Ahamad M, Mohamed M, Yean Yean C, et al. Loop-mediated isothermal amplification (LAMP) reaction as viable PCR substitute for diagnostic applications: a comparative analysis study of LAMP, conventional PCR, nested PCR (nPCR) and real-time PCR (qPCR) based on Entamoeba histolytica DNA derived from faecal sample. BMC Biotechnol. 2020; 20(1):34. Epub 20200622. doi: 10.1186/s12896-020-00629-8. PubMed PMID: 32571286; PubMed Central PMCID: PMCPMC7310076.
58. Waggoner J J, Rojas A, Mohamed-Hadley A, de Guillen Y A, Pinsky B A. Real-time RT-PCR for Mayaro virus detection in plasma and urine. J Clin Virol. 2018; 98:1-4. Epub 2017 Nov. 25. doi: 10.1016/j.jcv.2017.11.006. PubMed PMID: 29172075; PubMed Central PMCID: PMCPMC5742299.
59. Yoo H J, Baek C, Lee M H, Min J. Integrated microsystems for the in situ genetic detection of dengue virus in whole blood using direct sample preparation and isothermal amplification. Analyst. 2020; 145(6):2405-11. Epub 20200213. doi: 10.1039/c9an02435b. PubMed PMID: 32053125.
60. Kuboki N, Inoue N, Sakurai T, Di Cello F, Grab D J, Suzuki H, et al. Loop-mediated isothermal amplification for detection of African trypanosomes. J Clin Microbiol. 2003; 41(12):5517-24. doi: 10.1128/jcm.41.12.5517-5524.2003. PubMed PMID: 14662933; PubMed Central PMCID: PMCPMC308967.
61. Yang Q, Wang F, Prinyawiwatkul W, Ge B. Robustness of Salmonella loop-mediated isothermal amplification assays for food applications. J Appl Microbiol. 2014; 116(1):81-8. Epub 20131001. doi: 10.1111/jam. 12340. PubMed PMID: 24016159.
62. Yang Q, Chen S, Ge B. Detecting Salmonella serovars in shell eggs by loop-mediated isothermal amplification. J Food Prot. 2013; 76(10):1790-6. doi: 10.4315/0362-028x.jfp-13-140. PubMed PMID: 24112582.
63. Suleman E, Mtshali M S, Lane E. Investigation of false positives associated with loop-mediated isothermal amplification assays for detection of Toxoplasma gondii in archived tissue samples of captive felids. J Vet Diagn Invest. 2016; 28(5):536-42. Epub 20160722. doi: 10.1177/1040638716659864. PubMed PMID: 27449130.
64. Stridiron A K. Development and testing of LAMP assay for diagnosis of Plasmodium falciparum malaria. Ethnicity & Disease. 2009; 19(2 suppl. 3).
65. Francois P, Tangomo M, Hibbs J, Bonetti E J, Boehme C C, Notomi T, et al. Robustness of a loop-mediated isothermal amplification reaction for diagnostic applications. FEMS Immunol Med Microbiol. 2011; 62(1):41-8. Epub 20110316. doi: 10.1111/j. 1574-695X.2011.00785.x. PubMed PMID: 21276085.
66. Berth M, Delanghe J. Protein precipitation as a possible important pitfall in the clinical chemistry analysis of blood samples containing monoclonal immunoglobulins: 2 case reports and a review of the literature. Acta Clin Belg. 2004; 59(5):263-73. doi: 10.1179/acb.2004.039. PubMed PMID: 15641396.
67. Jankelow A M, Lee H, Wang W, Hoang T H, Bacon A, Sun F, et al. Smartphone clip-on instrument and microfluidic processor for rapid sample-to-answer detection of Zika virus in whole blood using spatial RT-LAMP. Analyst. 2022; 147(17):3838-53. Epub 20220822. doi: 10.1039/d2an00438k. PubMed PMID: 35726910; PubMed Central PMCID: PMCPMC9399074.
68. Curtis K A, Rudolph D L, Owen S M. Sequence-specific detection method for reverse transcription, loop-mediated isothermal amplification of HIV-1. J Med Virol. 2009; 81(6):966-72. doi: 10.1002/jmv.21490. PubMed PMID: 19382260.
69. Blohm G, Elbadry M A, Mavian C, Stephenson C, Loeb J, White S, et al. Mayaro as a Caribbean traveler: Evidence for multiple introductions and transmission of the virus into Haiti. Int J Infect Dis. 2019; 87:151-3. Epub 2019 Aug. 6. doi: 10.1016/j.ijid.2019.07.031. PubMed PMID: 31382049.
70. Brankatschk R, Bodenhausen N, Zeyer J, Bürgmann H. Simple absolute quantification method correcting for quantitative PCR efficiency variations for microbial community samples. Appl Environ Microbiol. 2012; 78(12):4481-9. Epub 20120406. doi: 10.1128/aem.07878-11. PubMed PMID: 22492459; PubMed Central PMCID: PMCPMC3370567.
71. Torezin Mendonça G, Cassaboni Stracke M, de Oliveira Coelho B, Bruna Soligo Sanchuki H, Klassen de Oliveira V, Klerynton Marchini F, et al. A new RT-LAMP-on-a-Chip Instrument for SARS-CoV-2 diagnostics. Microchem J. 2022; 180:107600. Epub 20220520. doi: 10.1016/j.microc.2022.107600. PubMed PMID: 35620142; PubMed Central PMCID: PMCPMC9121651.
72. Diagenetix. BioRanger detection kit.
73. Craw P, Balachandran W. Isothermal nucleic acid amplification technologies for point-of-care diagnostics: a critical review. Lab Chip. 2012; 12(14):2469-86. Epub 20120516. doi: 10.1039/c2lc40100b. PubMed PMID: 22592150.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, “about 0” can refer to 0, 0.001, 0.01, or 0.1. In an embodiment, the term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

1. An assay for the detection of the Mayaro virus (MAYV) comprising:
MAYV-specific primers comprising a MAYV-specific forward inner primer, a MAYV-specific backward inner primer, a MAYV-specific forward primer, and a MAYV-specific backward primer.

2. The assay according to claim 1, further comprising one or both of a MAYV-specific forward loop primer and a MAYV-specific backward loop primer.

3. The assay according to claim 1, wherein each of the MAYV-specific primers are complementary to nucleic acids encoding the NS1 nonstructural protein of MAYV.

4. The assay according to claim 1, wherein:
the MAYV-specific forward inner primer comprises a sequence having at least 90% sequence identity thereto the sequence AGACGCTCTGGGTCCTCAGCAATGATGTCTGAGCACACGT (SEQ ID NO:1); and
the MAYV-specific backward inner primer comprises a sequence having at least 90% sequence identity thereto the sequence
(SEQ ID NO: 2)
AGCCAAGGCATCAGGTGAAGTACTGACTGCAGGTCGTCTA.

5. The assay according to claim 2, wherein:
the MAYV-specific forward loop primer comprises a sequence having at least 75% sequence identity thereto the sequence CATTGGGCACACACAATGGT (SEQ ID NO:3).

6. The assay according to claim 2, wherein:
the MAYV-specific backward loop primer comprises a sequence having at least 75% sequence identity thereto the sequence CGTTGACAGAAATATTGCAGCAAAG (SEQ ID NO:4).

7. The assay according to claim 1, wherein:
the MAYV-specific forward primer comprises a sequence having at least 85% sequence identity thereto the sequence ACTCATCCTGGATATCGGCA (SEQ ID NO:5); and
the MAYV-specific backward primer comprises a sequence having at least 85% sequence identity thereto the sequence ACTCATTGTCCGGGGTCG (SEQ ID NO:6).

8. The assay according to claim 1, wherein:
the MAYV-specific forward inner primer comprising the sequence
(SEQ ID NO: 1)
AGACGCTCTGGGTCCTCAGCAATGATGTCTGAGCACACGT;
the MAYV-specific backward inner primer comprising the sequence
(SEQ ID NO: 2)
AGCCAAGGCATCAGGTGAAGTACTGACTGCAGGTCGTCTA;
the MAYV-specific forward loop primer comprising the sequence
(SEQ ID NO: 3)
CATTGGGCACACACAATGGT;
the MAYV-specific backward loop primer comprising the sequence
(SEQ ID NO: 4)
CGTTGACAGAAATATTGCAGCAAAG;
the MAYV-specific forward primer comprising the sequence ACTCATCCTGGATATCGGCA (SEQ ID NO:5); and
the MAYV-specific backward primer comprising the sequence ACTCATTGTCCGGGGTCG (SEQ ID NO:6).

9. The assay according to claim 1, wherein:
the MAYV-specific forward inner primer comprises a sequence having at least 90% sequence identity thereto the sequence TCGGTGCGTGAACTGCATAGACATCAGACATGCAGGACTCCA (SEQ ID NO:7); and
the MAYV-specific backward inner primer comprises a sequence having at least 90% sequence identity thereto the sequence
(SEQ ID NO: 8)
GGAGTACGCACAGCGTACTGGCCCCGGCCATTGTATCGA.

10. The assay according to claim 2, wherein:
the MAYV-specific forward loop primer comprises a sequence having at least 75% sequence identity thereto the sequence TGATAGACTGCCACCTCAGC (SEQ ID NO:9).

11. The assay according to claim 2, wherein:
the MAYV-specific backward loop primer comprises a sequence having at least 75% sequence identity thereto the sequence ATTGGGTTCGACACTACCCC (SEQ ID NO:10).

12. The assay according to claim 1, wherein:
the MAYV-specific forward primer comprises a sequence having at least 85% sequence identity thereto the sequence CGGACATTTTGCCTTCACAC (SEQ ID NO: 11); and
the MAYV-specific backward primer comprises a sequence having at least 85% sequence identity thereto the sequence GGCCCAGTTGGTTGCATAT (SEQ ID NO:12).

13. The assay according to claim 1, wherein:
the MAYV-specific forward inner primer comprising the sequence
(SEQ ID NO: 7)
TCGGTGCGTGAACTGCATAGACATCAGACATGCAGGACTCCA;
the MAYV-specific backward inner primer comprising the sequence
(SEQ ID NO: 8)
GGAGTACGCACAGCGTACTGGCCCCGGCCATTGTATCGA;
the MAYV-specific forward loop primer comprising the sequence
(SEQ ID NO: 9)
TGATAGACTGCCACCTCAGC;
the MAYV-specific backward loop primer comprising the sequence
(SEQ ID NO: 10)
ATTGGGTTCGACACTACCCC;
the MAYV-specific forward primer comprising the sequence CGGACATTTTGCCTTCACAC (SEQ ID NO:11); and
the MAYV-specific backward primer comprising the sequence
(SEQ ID NO: 12)
GGCCCAGTTGGTTGCATAT.

14. The assay according to claim 1, wherein at least one of the primers comprises a modification, wherein the modification is selected from a dye molecule, a conjugation molecule, a functional group, a molecular beacon, or a combination thereof.

15. The assay of claim 1, further comprising a mixture comprising one or more of an isothermal amplification buffer, a DNA polymerase, a reverse transcriptase, a concentrated primer mix, a deoxynucleotide triphosphate, MgSO₄, and nuclease-free water.

16. The assay of claim 15, further comprising Antarctic thermolabile uracil-DNA glycosylase and deoxyuridine triphosphate.

17. The assay of claim 15, wherein the assay is an RT-LAMP assay.

18. The assay of claim 15, wherein the total volume of the mixture is about 1 μL to about 1 mL.

19. The assay of claim 15, wherein the total volume of the mixture is about 25 μL.

20. A method of detecting Mayaro virus in a sample, the method comprising:
adding about 0.1 μL-10 mL of the sample to about 1 μL-1 mL of a RT-LAMP reaction mixture; and
amplifying RNA in the sample using RT-LAMP, wherein the sample contains from from 1 to 1×10⁷ genomic copies of Mayaro virus RNA per reaction.

21-27. (canceled)
